(19) United States
(12) Patent Application Publication
(10) Pub. No.: US 2012/0040855 A1
(43) Pub. Date: Feb. 16, 2012

(54) TISSUE-SPECIFIC AGING BIOMARKERS

(76) Inventors: Yuanlong Pan, Chesterfield, MO (US); Steven Scott Hannah, Chesterfield, MO (US); Rondo Paul Middleton, Creve Coeur, MO (US); Janet Rosann Jackson, Columbia, IL (US); Tomas Alberto Prolla, Madison, WI (US); Richard Howard Weindruch, Madison, WI (US); Jamie Louis Barger, Verona, WI (US); Thomas Darwin Pugh, Madison, WI (US)

(21) Appl. No.: 13/138,523

(22) PCT Filed: Mar. 10, 2010
(86) PCT No.: PCT/US10/00721

§ 371 Date: Oct. 26, 2011

Related U.S. Application Data
(62) Division of application No. 61/209,854, filed on Mar. 11, 2009.

(51) Int. Cl. C40B 30/04; C40B 40/10; C40B 40/06; C12Q 1/68; C40B 40/08

(52) U.S. Cl. 506/9; 435/6.1; 506/17; 506/16; 506/18; 435/6.12

(57) ABSTRACT

The invention provides methods of developing tissue-specific biomarkers of aging, sets of robust biomarkers identified by those methods, and uses of the biomarkers to identify nutrients and other functional ingredients or agents having anti-aging properties.

### CROSS REFERENCE TO RELATED APPLICATIONS

This application is a national stage application under 35 U.S.C. §371 of PCT/US2010/000721 filed Mar. 10, 2010, which claims priority to U.S. Provisional Application Ser. No. 61/209854 filed Mar. 11, 2009, the disclosures of which are incorporated herein by this reference.

### BACKGROUND OF THE INVENTION

1. Field of the Invention

The invention relates generally to the field of nutritional support of health and longevity in animals. In particular, the invention provides methods of developing tissue-specific universal biomarkers of aging in animals, as well as sets of robust biomarkers identified by those methods, and use of the tissue-specific universal aging biomarkers to identify nutrients and other functional ingredients or agents having anti-aging properties in animals.

2. Description of Related Art

Restriction of caloric intake well below ad libitum levels has been shown to increase lifespan, reduce or delay the onset of many age-related conditions, improve stress resistance and decelerate functional decline in numerous animal species, including mammals such as rodents and primates (See, e.g., D. K. Ingram et al. (2004) Ann. N.Y. Acad. Sci. 1019: 412-423). Indeed, clinical trials have been initiated to evaluate the longevity-promoting effect of caloric restriction (CR) in humans. But in humans and animals alike, it seems unlikely that CR is a viable strategy for increasing longevity in most individuals, due to the degree and length of restriction required. For this reason, research has focused on the identification of substances, e.g., pharmaceutical agents, nutritional substances and the like, capable of mimicking the effect of CR without a substantive change in dietary intake.

Efforts have been directed toward identifying agents that can mimic one or more of the physiological or biochemical effects of CR (See, e.g., Ingram et al., 2004, supra), or that can mimic the gene expression profile associated with CR in certain tissues and organs (e.g., Spindler, U.S. Pat. No. 6,406,853; U.S. Patent Pub. 2003/0124540). In connection with the latter, methods to analyze genes associated with CR and to screen for CR mimetics based on gene expression profiling have been disclosed (Spindler et al., U.S. Patent Pubs. 2004/0180003, 2004/0191775 and 2005/0013776; Pan et al., U.S. Patent Pub. 2007/0231371).

Despite the availability of the approaches summarized above, there remains a need for more robust, faster and less costly methods to screen for agents that can retard or reverse the aging process, to promote healthy aging and increase longevity. The present invention satisfies this need.

### SUMMARY OF THE INVENTION

It is, therefore, an object of the present invention to provide methods of identifying robust and universally applicable gene expression markers of aging in selected tissues, and to provide sets of robust and universally applicable gene expression markers identified by those methods.

It is another object of the invention to provide to provide one or more genes or gene segments that are differentially expressed in selected tissues of old subjects as compared with young subjects.

It is a further object of the invention to provide a combination comprising a plurality of polynucleotides that are differentially expressed in selected tissues of old subjects as compared with young subjects.

It is another object of the invention to provide compositions of two or more polynucleotide or polypeptide probes suitable for detecting the expression of genes differentially expressed in selected tissues of old subjects as compared with young subjects, and devices such as substrate arrays containing the probes.

It is a further object of the invention to provide methods for detecting differential expression of one or more genes differentially expressed in selected tissues of old subjects, as compared with young subjects or a standard reference.

It is another object of the invention to provide a method for measuring the effect of a test substance on the expression profile of one or more genes differentially expressed in selected tissues of old subjects, as compared with young subjects or a standard reference.

One or more of these other objects are achieved using novel methods of identifying tissue-specific aging biomarkers, and novel combinations of polynucleotides or polypeptides representing genes and gene segments that are differentially expressed in selected tissues of old subjects as compared with young subjects. The polynucleotides are used to produce compositions, probes, devices based on the probes, and methods for determining the status of polynucleotides differentially expressed in selected tissues of old subjects, as compared with young subjects or a standard reference, which are useful for achieving the above-identified objects, e.g., prognosing and diagnosing age-related conditions in selected tissues and for screening substances to determine if they are likely to have an anti-aging effect in a particular tissue. Various kits comprising combinations of probes, devices utilizing the probes, and substances are also provided, as are various computer programs for manipulating information, and communication media for communicating information pertaining to the differentially expressed genes and methods of their use.

Other and further objects, features, and advantages of the invention will be readily apparent to those skilled in the art.

### DETAILED DESCRIPTION OF THE INVENTION

### Definitions

As used throughout, ranges are used herein as shorthand, so as to avoid having to set out at length and describe each and every value within the range. Any appropriate value within the range can be selected, where appropriate, as the upper value, lower value, or the terminus of the range. It is understood that any and all whole or partial integers between any ranges or intervals set forth herein are included herein.

As used herein and in the appended claims, the singular form of a word includes the plural, and vice versa, unless the context clearly dictates otherwise. Thus, the references “a,” “an,” and “the” are generally inclusive of the plurals of the respective terms. For example, reference to “an animal”, “a method”, or “a substance” includes a plurality of such “animals”, “methods”, or “substances”. Similarly, the words “comprise”, “comprises”, and “comprising” are to be interpreted inclusively rather than exclusively.

The term “animal” means a human or other animal, including avian, bovine, canine, equine, feline, hicrine, murine, ovine, and porcine animals. When the term is used in the context of comparing test subjects, the animals that are compared are animals of the same species and possibly of the same race or breed. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Preferably, the animal is a human or a companion animal such as a canine or feline.

The term “antibody” means any immunoglobulin that binds to a specific antigen, including IgG, IgM, IgA, IgD, and IgE antibodies. The term includes polyclonal, monoclonal, monovalent, humanized, heteroconjugate, antibody compositions with polyepitopic specificity, chimeric, bispecific antibodies, diabodies, single-chain antibodies, and antibody fragments such as Fab, Fab′, F(ab′)₂, and Fv, or other antigen-binding fragments.

The term “array” means an ordered arrangement of at least two probes on a substrate. At least one of the probes is a control or standard and at least one of the probes is a diagnostic probe. The arrangement of from about two to about 40,000 probes on a substrate assures that the size and signal intensity of each labeled complex formed between a probe and a sample polynucleotide or polypeptide is individually distinguishable.

The term “binding complex” refers to a complex formed when a polypeptide in a sample specifically binds (as defined herein) to a binding partner, such as an antibody or functional fragment thereof.

The term “calorie restriction” or “caloric restriction” refers to any diet regimen low in calories without undernutrition. In general, the limitation is of total calories derived from of carbohydrates, fats, and proteins. The limitation is typically, although not limited to, about 25% to about 40% of the caloric intake relative to ad libitum consumption.

The term “dietary supplement” means a product that is intended to be ingested in addition to the normal diet of an animal. Dietary supplements may be in any form—e.g. solid, liquid, gel, tablets, capsules, powder, and the like. Preferably they are provided in convenient dosage forms. In some embodiments they are provided in bulk consumer packages such as bulk powders or liquids. In other embodiments, supplements are provided in bulk quantities to be included in other food items such as snacks, treats, supplement bars, beverages and the like.

The term “differential expression” or “differentially expressed” means increased or unregulated gene expression or means decreased or downregulated gene expression as detected by the absence, presence, or at least two-fold change in the amount of transcribed messenger RNA or translated protein in a sample.

The term “effective amount” means an amount of a compound, material, composition, medicament, or other material that is effective to achieve a particular biological result, such as reversing or delaying aging in a selected tissue, as described herein.

The term “food” or “food composition” means a composition that is intended for consumption by an animal, including a human, and provides nutrition thereto. As used herein, a “food product formulated for human consumption” is any composition specifically intended for ingestion by a human being. “Pet foods” are compositions intended for consumption by pets, preferably by companion animals. A “complete and nutritionally balanced pet food,” is one that contains all known required nutrients for the intended recipient or consumer of the food, in appropriate amounts and proportions, based for example on recommendations of recognized authorities in the field of companion animal nutrition. Such foods are therefore capable of serving as a sole source of dietary intake to maintain life or promote production, without the addition of supplemental nutritional sources. Nutritionally balanced pet food compositions are widely known and widely used in the art.

The term “fragment” means (1) an oligonucleotide or polynucleotide sequence that is a portion of a complete sequence and that has the same or similar activity for a particular use as the complete polynucleotide sequence or (2) a peptide or polypeptide sequence that is a portion of a complete sequence and that has the same or similar activity for a particular use as the complete polypeptide sequence. Such fragments can comprise any number of nucleotides or amino acids deemed suitable for a particular use. Generally, oligonucleotide or polynucleotide fragments contain at least about 10, 50, 100, or 1000 nucleotides and polypeptide fragments contain at least about 4, 10, 20, or 50 consecutive amino acids from the complete sequence. The term encompasses polynucleotides and polypeptides variants of the fragments.

The term “gene” or “genes” means a complete or partial segment of DNA involved in producing a polypeptide, including regions preceding and following the coding region (leader and trailer) and intervening sequences (introns) between individual coding segments (exons). The term encompasses any DNA sequence that hybridizes to the complement of gene coding sequences.

The term “gene product” means the product of transcription of a gene, such as mRNA or derivatives thereof (e.g., cDNA), or translation of a gene transcript. The term “gene product” generally refers to the translation product, which is a protein. The term “gene product” may be used interchangeably with the term “protein” herein.

The term “homolog” means (1) a polynucleotide, including polynucleotides from the same or different animal species, having greater than 30%, 50%, 70%, or 90% sequence similarity to a reference polynucleotide, and having the same or substantially the same properties and performing the same or substantially the same function as the reference polynucleotide, or having the capability of specifically hybridizing to a reference polynucleotide under stringent conditions or (2) a polypeptide, including polypeptides from the same or different animal species, having greater than 30%, 50%, 70%, or 90% sequence similarity to a reference polypeptide and having the same or substantially the same properties and performing the same or substantially the same function as the reference polypeptide, or having the capability of specifically binding to a reference polypeptide. When referring to fragments of full length coding sequences, the function of those fragments may simply be to encode a selected portion of a polypeptide of a certain sequence, or to be of suitably similar sequence to hybridize to another polynucleotide fragment encoding that polypeptide. When referring to fragments of polypeptides, the function of those fragments may simply be to form an epitope suitable for generation of an antibody. Sequence similarity of two polypeptide sequences or of two polynucleotide sequences is determined using methods known to skilled artisans, e.g., the algorithm of Karlin and Altschul (Proc. Natl. Acad. Sci. USA 87:2264-2268 (1990)). Such an algorithm is incorporated into the NBLAST and XBLAST programs of Altschul et al. (J. Mol. Biol. 215:403-410 (1990)). To obtain gapped alignments for comparison purposes, Gapped Blast can be utilized as described in Altschul et al. (Nucl. Acids Res. 25: 3389-3402 (1997)). When utilizing BLAST and Gapped BLAST programs, the default parameters of the respective programs (e.g., XBLAST and NBLAST) are used. See http://ww.ncbi.nlm.nih.gov.

The term “hybridization complex” means a complex that is formed between sample polynucleotides when the purines of one polynucleotide hydrogen bond with the pyrimidines of the complementary polynucleotide, e.g., 5′-A-G-T-C-3′ base pairs with 3′-T-C-A-G-5′. The degree of complementarily and the use of nucleotide analogs affect the efficiency and stringency of hybridization reactions.

The term “in conjunction” means that a drug, food, or other substance is administered to an animal (1) together in a composition, particularly food composition, or (2) separately at the same or different frequency using the same or different administration routes at about the same time or periodically. “Periodically” means that the substance is administered on a dosage schedule acceptable for a specific substance. “About the same time” generally means that the substance (food or drug) is administered at the same time or within about 72 hours of each other. “In conjunction” specifically includes administration schemes wherein substances such as drugs are administered for a prescribed period and compositions of the invention are administered indefinitely.

The term “individual” when referring to an animal means an individual animal of any species or kind. This term may be used interchangeably with the term “subject.”

The term “Longevity” refers generally to the duration of life beyond the average life expectancy for a particular species, or for a particular strain, breed or ethnic group within that species when distinctions within species exist. “Enhanced longevity” or “increased longevity” refers to any significant extension of the life span of a particular animal beyond the average life expectancy for the species to which the animal belongs.

The term “polynucleotide” or “oligonucleotide” means a polymer of nucleotides. The term encompasses DNA and RNA (including cDNA and mRNA) molecules, either single or double stranded and, if single stranded, its complementary sequence in either linear or circular form. The term also encompasses fragments, variants, homologs, and alleles, as appropriate for the sequences, which have the same or substantially the same properties and perform the same or substantially the same function as the original sequence. In particular, the term encompasses homologs from different species, e.g., a mouse and a dog or cat. The sequences may be fully complementary (no mismatches) when aligned or may have up to about a 30% sequence mismatch. Preferably, for polynucleotides, the chain contains from about 50 to 10,000 nucleotides, more preferably from about 150 to 3,500 nucleotides. Preferably, for oligonucleotides, the chain contains from about 2 to 100 nucleotides, more preferably from about 6 to 30 nucleotides. The exact size of a polynucleotide or oligonucleotide will depend on various factors and on the particular application and use of the polynucleotide or oligonucleotide. The term includes nucleotide polymers that are synthesized and that are isolated and purified from natural sources. The term “polynucleotide” is inclusive of “oligonucleotide.”

The term “polypeptide,” “peptide,” or “protein” means a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompass polymers of any length, preferably polymers containing from about 2 to 1000 amino acids, more preferably from about 5 to 500 amino acids. The term includes amino acid polymers that are synthesized and that are isolated and purified from natural sources.

The term “probe” means (1) an oligonucleotide or polynucleotide, either RNA or DNA, whether occurring naturally as in a purified restriction enzyme digest or produced synthetically, that is capable of annealing with or specifically hybridizing to a polynucleotide with sequences complementary to the probe or (2) a compound or substance, including a peptide or polypeptide, capable of specifically binding a particular protein or protein fragment to the substantial exclusion of other proteins or protein fragments. An oligonucleotide or polynucleotide probe may be either single or double stranded. The exact length of the probe will depend upon many factors, including temperature, source, and use. For example, for diagnostic applications, depending on the complexity of the target sequence, an oligonucleotide probe typically contains about 10 to 100, 15 to 50, or 15 to 25 nucleotides. In certain diagnostic applications, a polynucleotide probe contains about 100-1000, 300-600, nucleotides, preferably about 300 nucleotides. The probes herein are selected to be “substantially” complementary to different strands of a particular target sequence. This means that the probes must be sufficiently complementary to specifically hybridize or anneal with their respective target sequences under a set of predetermined conditions. Therefore, the probe sequence need not reflect the exact complementary sequence of the target. For example, a noncomplementary nucleotide fragment may be attached to the 5′ or 3′ end of the probe, with the remainder of the probe sequence being complementary to the target sequence. Alternatively, noncomplementary bases or longer sequences can be interspersed into the probe if the probe sequence has sufficient complementarity with the sequence of the target polynucleotide to specifically anneal to the target polynucleotide. A peptide or polypeptide probe may be any molecule to which the protein or peptide specifically binds, including DNA (for DNA binding proteins), antibodies, cell membrane receptors, peptides, cofactors, lectins, sugars, polysaccharides, cells, cell membranes, organelles and organellar membranes.

The term “sample” means any animal tissue or fluid containing, e.g., polynucleotides, polypeptides, antibodies, metabolites, and the like, including cells and other tissue containing DNA and RNA. Examples include adipose, blood, cartilage, connective, epithelial, lymphoid, muscle, nervous, sputum, and the like. A sample may be solid or liquid and may be DNA, RNA, cDNA, bodily fluids such as blood or urine, cells, cell preparations or soluble fractions or media aliquots thereof, chromosomes, organelles, and the like.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

The term “specifically bind” means a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

The term “specifically hybridize” means an association between two single stranded polynucleotides of sufficiently complementary sequence to permit such hybridization under predetermined conditions generally used in the art (sometimes termed “substantially complementary”). For example, the term may refer to hybridization of a polynucleotide probe with a substantially complementary sequence contained within a single stranded DNA or RNA molecule according to an aspect of the invention, to the substantial exclusion of hybridization of the polynucleotide probe with single stranded polynucleotides of non-complementary sequence.

The term “standard” means (1) a control sample that contains tissue from a subject administered a control or reference substance, or no substance, as compared with a sample that contains tissue from a subject administered a test substance, for example, to determine if the test substance causes differential gene expression, as appropriate for the context of its use.

The term “stringent conditions” means (1) hybridization in 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C., (2) hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C.; with washes at 42° C. in 0.2×SSC and 0.1% SDS or washes with 0.015 M NaCl, 0.0015 M sodium citrate, 0.1% Na2SO4 at 50° C. or similar procedures employing similar low ionic strength and high temperature washing agents and similar denaturing agents.

The term “tissue-specific marker” or “tissue-specific biomarker” as used herein refers to genes and their expression products that are differentially expressed in a selected tissue of an old, as compared with young, subject. The term “tissue-specific” is intended to encompass tissues and organs. For example, the selected tissue may be smooth muscle tissue from the heart, and the tissue specific markers may be referred to as “heart-specific.” As another example, the selected tissue may be adipose tissue, which may not be associated with any particular organ. The skilled artisan will understand these terms as they are used in context throughout the specification.

The term “variant” means (1) a polynucleotide sequence containing any substitution, variation, modification, replacement, deletion, or addition of one or more nucleotides from or to a polynucleotide sequence and that has the same or substantially the same properties and performs the same or substantially the same function as the original sequence and (2) a polypeptide sequence containing any substitution, variation, modification, replacement, deletion, or addition of one or more amino acids from or to a polypeptide sequence and that has the same or substantially the same properties and performs the same or substantially the same function as the original sequence. The term therefore includes single nucleotide polymorphisms (SNPs) and allelic variants and includes conservative and non-conservative amino acid substitutions in polypeptides. The term also encompasses chemical derivatization of a polynucleotide or polypeptide and substitution of nucleotides or amino acids with nucleotides or amino acids that do not occur naturally, as appropriate.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

“Young” refers generally to an individual in young adulthood, i.e., matured past puberty or adolescence, as would be defined by species, or by strain, breed or ethnic group within a species, in accordance with known parameters. “Aged” or “old,” as used herein, refers to an individual who is physically or chronologically within the last 30% of its average life expectancy, as determined by species, or by strain, breed or ethnic group within a species, in accordance with known parameters.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to and does not limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, and other references cited or referred to herein are incorporated herein by reference to the extent allowed by controlling law. The discussion of those references is intended merely to summarize the assertions made therein. No admission is made that any such patents, patent applications, publications or references, or any portion thereof, is relevant, material, or prior art. The right to challenge the accuracy and pertinence of any assertion of such patents, patent applications, publications, and other references as relevant, material, or prior art is specifically reserved.

### The Invention

The present invention arises in part from the inventors' development of a method for identifying robust gene expression markers of aging in selected tissues. The method involves the step of screening for differential gene expression in selected tissues in a plurality of strains, breeds or ethnic groups in a species, and employs a criterion that a candidate gene expression marker must be differentially expressed in a majority of the strains, breeds or ethnic groups that are screened. Using this and, optionally, one or more secondary screening criteria, robust sets of gene expression markers of aging in several selected tissues have been identified.

In certain embodiments of the invention, the markers are used to measure expression of at least one differentially expressed gene. In preferred embodiments, the markers are used to measure expression of two or more differentially expressed genes. Measuring two or more differentially expressed genes provides a gene expression pattern or gene expression profile for the selected tissue. More preferably, measurement of a multiplicity of differentially expressed genes in several selected tissues may be performed, providing additional information for a gene expression pattern or profile.

In various embodiments of the invention, changes in gene expression may be measured in one or both of two ways: (1) measuring transcription through detection of mRNA produced by a particular gene; and (2) measuring translation through detection of protein produced by a particular transcript.

Decreased or increased expression can be measured at the RNA level using any of the methods well known in the art for the quantitation of polynucleotides, such as, for example, PCR (including, without limitation, RT-PCR and qPCR), RNase protection, Northern blotting, microarray, macroarray, and other hybridization methods. The genes that are assayed or interrogated according to the invention are typically in the form of mRNA or reverse transcribed mRNA. The genes may be cloned and/or amplified. The cloning itself does not appear to bias the representation of genes within a population. However, it may be preferable to use polyA+ RNA as a source, as it can be used with fewer processing steps.

Thus, in one aspect, the invention provides methods for identifying gene expression markers of aging in a selected tissue. The methods comprise selecting one or more genes differentially expressed in the tissue in old subjects as compared with young subjects, using a criterion that the gene is differentially expressed in the selected tissues in a multiplicity of strains, breeds or ethnic groups of a species, preferably at a pre-determined significance level (e.g., p<0.10, p<0.05, or p<0.01). In certain embodiments, the gene is differentially expressed in, 2, 3, 4, 5, 6, 7, 8, 9 or 10 or more strains, breeds or ethnic groups. In other embodiments, the criterion is set that the gene is differentially expressed in a majority of the strains, breeds or ethnic groups tested, and can be elevated such that the gene should be differentially expressed in at least 50%, 60%, 70%, 80%, 90% or 100% of the strains, breeds or ethnic groups tested.

The method can be practiced on strains, breeds or ethnic groups of any species. In particular embodiments, the species is a mammal, and in particular a human or a companion animal, such as a canine or feline, or other companion animals as defined above.

The tissue selected for practice of the method may be any tissue or organ, including but not limited to adipose, bladder, blood, bone, bone marrow, bowel, brain and central nervous system, breast, bronchus, cartilage, colo-rectal, connective tissue, endocrine system, eye, female reproductive organs, glands, heart, intestine, kidney, liver, lung and nasal/bronchial system, lymph node and lymphoid organs, male reproductive organs, mouth and tongue, neural tissue other than brain/CNS, pancreas, peritoneum, spleen, and stomach, to name a few. In exemplary embodiments, the tissue is selected from heart, muscle, brain or adipose tissue.

The method described above can include further criteria for identifying robust markers of aging in selected tissues. For example, the method can further comprise a criterion that differential expression of the gene differentially expressed in old subjects as compared with young subjects is at least partially reversed by caloric restriction. The method can also further comprise a criterion that the gene differentially expressed in old subjects as compared with young subjects is known or suspected to be associated with one or more aging-related physiological functions. The functionality of a gene product can be determined from experimentation or from the literature available to the skilled artisan.

The methods described above are used to identify biomarkers of aging in selected tissues. Accordingly, in another aspect, the invention provides combinations comprising a plurality of polynucleotides or proteins expressed therefrom that are differentially expressed in selected tissues of old subjects as compared with young subjects, wherein the polynucleotides are selected from genes encoding proteins listed in Table 2, Table 5, Table 8 or Table 10, or fragments thereof. These tables list gene designations, gene names and “Entrez” numbers, which enable access to the full description of the genes and gene products in the National Institutes of Health National Center for Biotechnology Information (NCBI) database.

In one embodiment, the selected tissue is heart and the polynucleotides are selected from genes encoding two or more of Amy1, Apod, Bdh1, C3, Casq1, Ce18, Kcnd2, Lcn2, Mt2, Myot, Pah, Prkeq, Serpina3n, Skap2, Tmeml6k, and Vllg2. Of this group the differential expression is reversed by caloric restriction in C3, Cc18, Lcn2, Mt2, Pah, Prkcq, Serpina3n, Tmeml6k, and Vgll2.

In another embodiment, the selected tissue is adipose and the polynucleotides are selected from genes encoding two or more of Aspn, Clec4n, Col6a2, Coll8a1, Cox8b, Crip2, Ear11, Emilin2, Otop1, Pla2g2d, Rhbd13, Slc6a13, and Sycp3. Of this group, the differential expression is reversed by caloric restriction in Aspn, Col6a2, Crip2, Emilin2, Otop1, Pla2g2d, Rhbd13, and Slc6a13.

In another embodiment, the selected tissue is brain and the polynucleotides are selected from genes encoding two or more of Apod, B2m, Clqa, Clqb, Cd68, Clec7a, Cst7, Ctsd, Gfap, Il33, Lgals3, Lyzs, and Spp1. Of this group, the differential expression is reversed by caloric restriction in Apod, B2m, Clqa, Clqb, Ctsd, Gfap, Il33, Lyzs, and Spp1.

In another embodiment, the selected tissue is muscle and the polynucleotides are selected from genes encoding two or more of C4, Cdkn2c, Cds1, Colla1, Col1a2, Col3a1, Dusp26, Edg2, Igh-6, Mt2, Plk2, Rhpn2, and Syt9. Of this group, the differential expression is reversed by caloric restriction in C4, Cdkn2c, Cds1, Colla1, Col1a2, Col3a1, Edg2, Igh-6, Mt2, Plk2, and Syt9.

In one embodiment, the combination comprises two or more polynucleotides or proteins expressed from the polynucleotides. Preferably, the combination comprises a plurality of polynucleotides or proteins expressed from polynucleotides, generally about 3, 4, 5, 6, 7, 8, 9, 10, 11, 12, 13, 14, 15, 16, 17, 18, 19, 20, 25, 30, 35, 40, 45, 50, 60, 70, 80, 90, 100 or more polynucleotides or proteins, or fragments thereof, as appropriate for a particular species, tissue and use. When the combination comprises one or more fragments, the fragments can be of any size that retains the properties and function of the original polynucleotide or protein, preferably from about 30%, 60%, or 90% of the original.

The polynucleotides and proteins can be from any animal including humans, particularly canines and felines, most particularly canines. Homologs of the polynucleotides and proteins from different animal species are obtainable by standard information mining and molecular methods well known to the skilled artisan. For example, the name, public database accession number, or description of function of a gene or protein may be entered into one of several publicly available databases, which will generate a list of sources providing information about that gene from different species, including sequence information. One such database is the “Information Hyperlinked over Proteins (iHOP) database, which is accessible on the interne via the url: ihop-net.org. Alternatively, a public database accession number of a known gene or protein may be utilized to access sequence information for that gene or protein and to search for homologs or orthologs in other species using a sequence comparison search. For example, the GenBank accession number of a gene or protein from mouse may be entered into the National Institutes of Health's National Center for Biotechnology Information (NCBI) database, thereby accessing DNA or polypeptide sequences for that mouse gene. Using the same database, a BLAST search may be performed on the mouse DNA or protein sequence, or fragments thereof of sufficient length to define the gene or protein, to identify sequences of sufficient homology from other species, e.g., a canine. Accession numbers of the sequences from the other species of interest may then be entered into the database to obtain information pertaining to those full-length nucleotide or protein sequences, as well as other descriptive information.

In another aspect, the invention provides compositions comprising two or more probes for detecting differential gene expression in a selected tissue in old subjects as compared with young subjects. In certain embodiments, the selected tissue is heart, adipose, brain or muscle tissue, and the probes comprise: (a) polynucleotides that specifically hybridize to two or more genes encoding proteins listed in Table 2, Table 5, Table 8 or Table 10, or fragments thereof; or (b) polypeptide binding agents that specifically bind to two or more polypeptides selected from proteins listed in Table 2, Table 5, Table 8 or Table 10, or fragments thereof.

In one embodiment, the selected tissue is heart, and the proteins encoded by the differentially expressed genes are Amy1, Apod, Bdh1, C3, Casq1, Cc18, Kcnd2, Lcn2, Mt2, Myot, Pah, Prkcq, Serpina3n, Skap2, Tmeml6k, and Vgll2. In another embodiment, the selected tissue is adipose and the proteins encoded by the differentially expressed genes are Aspn, Clec4n, Col6a2, Coll8a1, Cox8b, Crip2, Emilin2, Otop1, Pla2g2d, Rhbdl3, Slc6a13, and Sycp3. In another embodiment, the selected tissue i sbrain and the proteins encoded by the differentially expressed genes are Apod, B2m, Clqa, Clqb, Cd68, Clec7a, Cst7, Ctsd, Gfap, Il33, Lgals3, Lyzs, and Spp1. In still another embodiment, the selected tissue is muscle and the proteins encoded by the differentially expressed genes are C4, Cdkn2c, Cds1, Colla1, Colla2, Col3a1, Dusp26, Edg2, Igh-6, Mt2, Plk2, Rhpn2, and Syt9.

In particular embodiments, the differential expression is reversed by caloric restriction and the proteins encoded by the differentially expressed genes are: (a) C3, Cc18, Lcn2, Mt2, Pah, Prkcq, Serpina3n, Tmeml6k, and Vgll2 in the heart; (b) Aspn, Col6a2, Crip2, Emilin2, Otop1, Pla2g2d, Rhbd13, and Slc6a13 in adipose tissue; (c) Apod, B2m, Clqa, Clqb, Ctsd, Gfap, Il33, Lyzs, and Spp 1 in the brain; or (d) C4, Cdkn2c, Cds1, Colla1, Colla2, Col3a1, Edg2, Igh-6, Mt2, Plk2, and Syt9 in muscle.

Preferably, the composition comprises a plurality of probes, generally about 5, 10, 15, 20, 25, 30, 35, 40, 45, 50, 60, 70, 80, 90, 100, 200, 500 or more probes for detecting the polynucleotides or proteins, or fragments thereof, as appropriate for a particular species, tissue and use. It Will be understood by the skilled artisan that multiple different probes for a single target gene or protein may be utilized, to refine the sensitivity or accuracy of an assay utilizing the probes. For example, several oligonucleotide probes, specifically hybridizing to different sequences on a target polynucleotide, may be employed. Likewise, several antibodies, immunologically specific for different epitopes on a target protein, may be utilized.

One or more oligonucleotide or polynucleotide probes for interrogating a sample may be prepared using the sequence information for any of the genes listed herein, from any species, preferably canine or feline. The probes should be of sufficient length to specifically hybridize substantially exclusively with appropriate complementary genes or transcripts. In certain embodiments, the oligonucleotide probes will be at least about 10, 12, 14, 16, 18, 20 or 25 nucleotides in length. In some embodiments, longer probes of at least about 30, 40, 50, 60, 70, 80, 90 or 100 nucleotides are desirable, and probes longer than about 100 nucleotides may be suitable in some embodiments. The probes may comprise full length sequences encoding functional proteins. The nucleic acid probes are made or obtained using methods known to skilled artisans, e.g., in vitro synthesis from nucleotides, isolation and purification from natural sources, or enzymatic cleavage of the polynucleotides of the invention.

Hybridization complexes comprising nucleic acid probes hybridized to a polynucleotide of the invention may be detected by a variety of methods known in the art. In certain embodiments of the invention, immobilized nucleic acid probes may be used for the rapid and specific detection of polynucleotides and their expression patterns. Typically, a nucleic acid probe is linked to a solid support and a target polynucleotide (e.g., a gene, a transcription product, an amplicon, or, most commonly, an amplified mixture) is hybridized to the probe. Either the probe, or the target, or both, can be labeled, typically with a fluorophore or other tag, such as streptavidin. Where the target is labeled, hybridization may be detected by detecting bound fluorescence. Where the probe is labeled, hybridization is typically detected by quenching of the label. Where both the probe and the target are labeled, detection of hybridization is typically performed by monitoring a color shift resulting from proximity of the two bound labels. A variety of labeling strategies, labels, and the like, particularly for fluorescent based applications, are known in the art.

In another embodiment, the probes comprise polypeptide binding agents that specifically bind to polypeptides produced by expression of one or more of the polypeptides listed herein, or fragments thereof. Such protein binding probes may be prepared using the sequence information available for any of the proteins identified in Table 2, Table 5, Table 8 and Table 10, or fragments thereof.

Assay techniques that can be used to determine levels of a protein in a sample are also well known to those of skill in the art. Such assay methods include radioimmunoassays, competitive-binding assays, Western blot analysis and ELISA assays. In the assay methods utilizing antibodies, both polyclonal and monoclonal antibodies are suitable for use in the invention. Such antibodies may be immunologically specific for a particular protein, or an epitope of the protein, or a protein fragment, as would be well understood by those of skill in the art. Methods of making polyclonal and monoclonal antibodies immunologically specific for a protein or peptide are also well known in the art.

Preferred embodiments of the invention may utilize antibodies for the detection and quantification of proteins produced by expression of the genes described herein. Though proteins may be detected by immunoprecipitation, affinity separation, Western blot analysis and the like, a preferred method utilizes ELISA-type methodology wherein the antibody is immobilized on a solid support and a target protein or peptide is exposed to the immobilized antibody. Either the probe, or the target, or both, can be labeled. A variety of labeling strategies, labels, and the like, are known in the art.

In another aspect, the invention provides devices comprising a solid support to which is affixed an array comprising a plurality of probes for detecting differential gene expression in a selected tissue in old subjects as compared with young subjects. In certain embodiments, the selected tissue is heart, adipose, brain or muscle tissue, and the probes comprise: (a) polynucleotides that specifically hybridize to two or more genes encoding proteins listed in Table 2, Table 5, Table 8 or Table 10, or fragments thereof; or (b) polypeptide binding agents that specifically bind to two or more polypeptides selected from proteins listed in Table 2, Table 5, Table 8 or Table 10, or fragments thereof. In a preferred embodiment, the device is uses to detect differential expression of genes from canines or felines.

In one embodiment, the selected tissue is heart, and the proteins encoded by the differentially expressed genes are Amy1, Apod, Bdh1, C3, Casq1, Cc18, Kcnd2, Lcn2, Mt2, Myot, Pah, Prkcq, Serpina3n, Skap2, Tmeml6k, and Vgll2. In another embodiment, the selected tissue is adipose and the proteins encoded by the differentially expressed genes are Aspn, Clec4n, Col6a2, Coll8a1, Cox8b, Crip2, Ear11, Emilin2, Otop1, Pla2g2d, Rhbdl3, Slc6a13, and Sycp3. In another embodiment, the selected tissue is brain and the proteins encoded by the differentially expressed genes are Apod, B2m, Clqa, Clqb, Cd68, Clec7a, Cst7, Ctsd, Gfap, Il33, Lgals3, Lyzs, and Spp1. In still another embodiment, the selected tissue is muscle and the proteins encoded by the differentially expressed genes are C4, Cdkn2c, Cds1, Colla1, Colla2, Col3a1, Dusp26, Edg2, Igh-6, Mt2, Plk2, Rhpn2, and Syt9.

In particular embodiments, the differential expression is reversed by caloric restriction and the proteins encoded by the differentially expressed genes are: (a) C3, Cc18, Lcn2, Mt2, Pah, Prkcq, Serpina3n, Tmeml6k, and Vgll2 in the heart; (b) Aspn, Col6a2, Crip2, Emilin2, Otop1, Pla2g2d, Rhbd13, in adipose tissue; (c) Apod, B2m, Clqa, Clqb, Ctsd, Gfap, Il33, Lyzs, and Spp1 in the brain; or (d) C4, Cdkn2c, Cds1, Colla1, Colla2, Col3a1, Edg2, Igh-6, Mt2, Plk2, and Syt9 in muscle.

In one embodiment, arrays of oligonucleotide or polynucleotide probes may be utilized, whereas another embodiment may utilize arrays of antibodies or other proteins that bind specifically to the differentially expressed gene products. Such arrays may be custom made according to known methods, such as, for example, in-situ synthesis on a solid support or attachment of pre-synthesized probes to a solid support via micro-printing techniques. In preferred embodiments, arrays of nucleic acid or protein-binding probes are custom made to specifically detect transcripts or proteins produced by two or more of the differentially expressed genes or gene fragments described herein.

In another aspect, the invention provides methods for detecting differential expression of one or more genes differentially expression in a selected tissue in old subjects as compared with a standard or with young subjects. In particular embodiments, the tissue is heart, adipose, brain or muscle, and the methods generally comprise: (a) providing probes comprising (i) polynucleotides that specifically hybridize to two or more genes encoding proteins listed in Table 2, Table 5, Table 8 or Table 10, or fragments thereof; or (ii) polypeptide binding agents that specifically bind to two or more polypeptides selected from proteins listed in Table 2, Table 5, Table 8 or Table 10, or fragments thereof; (b) adding the probes to a sample comprising mRNA or proteins from an old subject, in a manner enabling hybridization or binding of the probes to the mRNA or proteins in the sample, thereby forming hybridization or binding complexes in the sample; (c) optionally, adding the probes to another sample comprising mRNA or proteins from a young subject, in a manner enabling hybridization or binding of the probes to the mRNA or proteins in the second sample, thereby forming hybridization or binding complexes in the other sample; (d) detecting the hybridization complexes in the sample or samples; and (e) comparing the hybridization or binding complexes from the first sample with the hybridization or binding complexes from a standard or, optionally, from the other sample, wherein at least one difference between the amount of hybridization or binding in the sample as compared with the standard or the optional other sample indicates differential expression of the one or more genes differentially expressed in the old subjects.

The method may be used to detect differential expression of genes encoding the gene products set forth in Tables 2, 5, 8 or 10, or in subsets thereof. Thus, in one embodiment, the selected tissue is heart, and the proteins encoded by the differentially expressed genes are Amy1, Apod, Bdh1, C3, Casq1, Cc18, Kcnd2, Lcn2, Mt2, Myot, Pah, Prkcq, Serpina3n, Skap2, Tmeml6k, and Vgll2. In another embodiment, the selected tissue is adipose and the proteins encoded by the differentially expressed genes are Aspn, Clec4n, Col6a2, Coll8a1, Cox8b, Crip2, Ear11, Emilin2, Otop1, Pla2g2d, Rhbdl3, Slc6a13, and Sycp3. In another embodiment, the selected tissue is brain and the proteins encoded by the differentially expressed genes are Apod, B2m, Clqa, Clqb, Cd68, Clec7a, Cst7, Ctsd, Gfap, Il33, Lgals3, Lyzs, and Spp1. In still another embodiment, the selected tissue is muscle and the proteins encoded by the differentially expressed genes are C4, Cdkn2c, Cds1, Colla1, Colla2, Col3a1, Dusp26, Edg2, Igh-6, Mt2, Plk2, Rhpn2, and Syt9.

In particular embodiments, the differential expression is reversed by caloric restriction and the proteins encoded by the differentially expressed genes are: (a) C3, Cc18, Lcn2, Mt2, Pah, Prkcq, Serpina3n, Tmeml6k, and Vgll2 in the heart; (b) Aspn, Col6a2, Crip2, Emilin2, Otop1, Pla2g2d, Rhbd13, and Slc6a13 in adipose tissue; (c) Apod, B2m, Clqa, Clqb, Ctsd, Gfap, Il33, Lyzs, and Spp 1 in the brain; or (d) C4, Cdkn2c, Cds1, Colla1, Colla2, Col3a1, Edg2, Igh-6, Mt2, Plk2, and Syt9 in muscle.

In a preferred embodiment, the method is uses to detect differential expression of genes from canines or felines. In particular embodiments, the probes are bound to a substrate, preferably in an array.

Step (c) and part of steps (d) and (e) are optional and are used if a relatively contemporaneous comparison of two or more test systems (i.e., tissues from old and young subjects) is to be conducted. However, in another embodiment, the standard used for comparison is based upon data previously obtained using the method. In this embodiment, the probes are exposed to a sample to form hybridization or binding complexes that are detected and compared with those of a standard. The differences between the hybridization or binding complexes from the sample and standard indicate differential expression of polynucleotides and therefore genes differentially expressed in tissue of the old subject versus the standard, which can comprise mRNA previously isolated from a young subject or another type of reference subject. In a preferred embodiment, probes are made to specifically detect polynucleotides or fragments thereof produced by one or more of the genes or gene fragments identified by the invention. Methods for detecting hybridization complexes are known to skilled artisans.

The assays described herein utilizing tissue-specific biomarkers for the detection of aging related transcription and translation products are useful in methods for determining the physiological age of a tissue in a subject. Such methods may be useful for implementing, facilitating, or guiding an anti-aging regimen, such as caloric restriction and/or a nutritional regimen. Such methods comprise obtaining a sample of the selected tissue from a subject undergoing such a regimen. The tissue sample is then analyzed for modulated expression of one or more genes associated with a young versus old phenotype, using a gene- or protein-array or other detection method as described herein. The results of the analysis will reveal whether the regimen is effective in delaying or reversing the aging process in the tissue.

In another aspect, the invention provides methods of determining if a test substance is likely to be useful in reversing or delaying the aging process in at least one selected tissue when administered to an animal. The methods comprise (a) determining a first gene expression profile by measuring the transcription or translation products of two or more polynucleotides selected from genes encoding proteins listed in Table 2, Table 5, Table 8 or Table 10, or fragments thereof, in a test system in the absence of the test substance; (b) determining a second gene expression profile by measuring the transcription or translation products of two or more polynucleotides selected from genes encoding proteins listed in Table 2, Table 5, Table 8 or Table 10, or fragments thereof, in a test system in the presence of the test substance; and (c) comparing the first gene expression profile with the second gene expression profile, wherein a change in the second gene expression profile as compared with the first gene expression profile indicates that the test substance is likely to be useful in reversing or delaying the aging process when administered to an animal. When comparing the first gene expression profile with the second gene expression profile, the comparison can be either at the individual transcription or translation product level or as an average of the aging changes for all transcription or translation products. This method is useful for generating an aging retardation index.

In one embodiment, the selected tissue is heart, and the proteins encoded by the differentially expressed genes are Amy1, Apod, Bdh1, C3, Casq1, Ccl8, Kcnd2, Lcn2, Mt2, Myot, Pah, Prkcq, Serpina3n, Skap2, Tmem16k, and Vgll2. In another embodiment, the selected tissue is adipose and the proteins encoded by the differentially expressed genes are Aspn, Clec4n, Col6a2, Col18a1, Cox8b, Crip2, Ear11, Emilin2, Otop1, Pla2g2d, Rhbdl3, Slc6a13, and Sycp3. In another embodiment, the selected tissue is brain and the proteins encoded by the differentially expressed genes are Apod, B2m, Clqa, Clqb, Cd68, Clec7a, Cst7, Ctsd, Gfap, Il33, Lgals3, Lyzs, and Spp1. In still another embodiment, the selected tissue is muscle and the proteins encoded by the differentially expressed genes are C4, Cdkn2c, Cds1, Colla1, Colla2, Col3a1, Dusp26, Edg2, Igh-6, Mt2, Plk2, Rhpn2, and Syt9.

In particular embodiments, the differential expression is reversed by caloric restriction and the proteins encoded by the differentially expressed genes are: (a) C3, Cc18, Lcn2, Mt2, Pah, Prkcq, Serpina3n, Tmeml6k, and Vgll2 in the heart; (b) Aspn, Col6a2, Crip2, Emilin2, Otop1, Pla2g2d, Rhbd13, and Slc6a13, in adipose tissue; (c) Apod, B2m, Clqa, Clqb, Ctsd, Gfap, Il33, Lyzs, and Spp 1 in the brain; or (d) C4, Cdkn2c, Cds1, Colla1, Colla1, Col3a1, Edg2, Igh-6, Mt2, Plk2, and Syt9 in muscle.

In certain embodiments, the method may further include the step of comparing at least the second gene expression profile with a reference or standard gene expression profile obtained by measuring the transcription or translation products of two or more polynucleotides selected from genes encoding proteins listed in Tables 2, 5, 8 or 10, or fragments thereof, in a test system in the presence of a reference substance or composition known reverse or delay aging in a particular tissue or tissues when administered to animals.

In one embodiment, the test system comprises a population of cultured cells. A nucleic acid construct comprising an aging-related gene according to the invention is introduced into cultured host cells. The host cells can be mammalian cell lines, such as but are not limited, to NIH3T3, CHO, HELA, and COS, although non-mammalian cells such as yeast, bacteria and insect cells can also be used. The coding sequences of the genes are operably linked to appropriate regulatory expression elements suitable for the particular host cell to be utilized. The nucleic acid constructs can be introduced into the host cells according to any acceptable means in the art, including but not limited to, transfection, transformation, calcium phosphate precipitation, electroporation and lipofection. Such techniques are well known and routine in the art. Transformed cells can be also used to identify compounds that modulate expression of the aging-related genes.

Gene expression assays can be carried out using a gene construct comprising the promoter of a selected aging-related gene operably linked to a reporter gene. The reporter construct may be introduced into a suitable cultured cell, including, without limitation, the standard host cell lines described above, or cells freshly isolated from a subject, such as adipose or muscle cells. The assay is performed by monitoring expression of the reporter gene in the presence or absence of a test compound.

In a preferred embodiment, the test system comprises animals. Typically, a test compound is administered to a subject and the gene expression profile in a selected tissue of the subject is analyzed to determine the effect of the test compound on transcription or the translation of the aging-related genes or gene products of the invention. Gene expression can be analyzed in situ or ex vivo to determine the effect of the test compound. In another embodiment, a test compound is administered to a subject and the activity of a protein expressed from a gene is analyzed in situ or ex vivo according to any means suitable in the art to determine the effect of the test compound on the activity of the proteins of interest. In addition, where a test compound is administered to a subject, the physiological, systemic, and physical effects of the compound, as well as potential toxicity of the compound can also be evaluated.

Test substances can be any substance and combination of substances that may have an effect on polynucleotides or genes differentially expressed in selected tissues of old versus young subjects. Suitable test substances include, but are not limited to, amino acids; proteins, peptides, polypeptides, nucleic acids, oligonucleotides, polynucleotides, small molecules, macromolecules, vitamins, minerals, simple sugars; complex sugars; polysaccharides; carbohydrates; medium-chain triglycerides (MCTs); triacylglycerides (TAGs); n-3 (omega-3) fatty acids including DHA, EPA, ALA; n-6 (omega-6) fatty acids including LA, γ-linolenic acid (GLA) and ARA; SA, conjugated linoleic acid (CLA); choline sources such as lecithin; fat-soluble vitamins including vitamin A and precursors thereof such as carotenoids (e.g., (n-carotene), vitamin D sources such as vitamin D2 (ergocalciferol) and vitamin D3 (cholecalciferol), vitamin E sources such as tocopherols (e.g., a-tocopherol) and tocotrienols, and vitamin K sources such as vitamin K1 (phylloquinone) and vitamin K2 (menadione); water-soluble vitamins including B vitamins such as riboflavin, niacin (including nicotinamide and nicotinic acid), pyridoxine, pantothenic acid, folic acid, biotin and cobalamin; and vitamin C (ascorbic acid); antioxidants, including some of the vitamins listed above, especially vitamins E and C; also bioflavonoids such as catechin, quercetin and theaflavin; quinones such as ubiquinone; carotenoids such as lycopene and lycoxanthin; resveratrol; and a-lipoic acid; L-carnitine; D-limonene; glucosamine; S-adenosylmethionine; and chitosan. In a preferred embodiment, test substances are nutrients that may be added to food or consumed as a supplement. Substances identified by the foregoing method are also contemplated as part of the invention.

In another aspect, the invention provides kits comprising, in separate containers in a single package, or in separate containers in a virtual package, two or more probes for detecting differential gene expression in a selected tissue in old subjects as compared with young subjects. In certain embodiments, the tissue is heart, adipose, brain or muscle tissue and the probes comprise (a) polynucleotides that specifically hybridize to two or more genes encoding proteins listed in Table 2, Table 5, Table 8 or Table 10, or fragments thereof; or (b) polypeptide binding agents that specifically bind to two or more polypeptides selected from proteins listed in Table 2, Table 5, Table 8 or Table 10, or fragments thereof; wherein the kit further comprises at least one of (1) instructions for how to use the probes in a gene expression assay for detecting differential gene expression in selected tissues of subjects, (2) reagents and equipment for using the probes, and (3) a composition known to reverse or delay the aging process in the selected tissue when administered to the subject.

When the kit comprises a virtual package, the kit is limited to instructions in a virtual environment in combination with one or more physical kit components. In one embodiment, the kit contains probes and/or other physical components and the instructions for using the probes and other components are available via the interne. The kit may contain additional items such as a device for mixing samples, probes, and reagents and device for using the kit, e.g., test tubes or mixing utensils.

In another aspect, the invention provides computer systems comprising a database containing information about polynucleotides that are differentially expressed in a selected tissue of old subjects as compared with young subjects. The database can contain information identifying the expression level of one or more polynucleotides selected from genes encoding proteins listed in Tables 2, 5, 8 or 10, and/or polypeptides that specifically bind to the proteins listed in Tables 2, 5, 8 or 10, and a user interface to interact with the database, particularly to input, manipulate, and review the information for different animals or categories of animals. In one embodiment, the database further contains information identifying the activity level of one or more polypeptides listed in Tables 2, 5, 8 or 10. In another, the database further comprises sequence information for one or more of the polynucleotides or polypeptides as listed in Tables 2, 5, 8 or 10, preferably from a variety of species. In other embodiments, the database contains additional information pertaining to the description of the genes in one or more animal species. The computer system is any electronic device capable of containing and manipulating the data and interacting with a user, e.g., a typical computer or an analytical instrument designed to facilitate using the invention and outputting the results relating to the status of an animal.

In another aspect, the invention provides media for communicating information about or instructions for one or more of compositions and methods described herein. Such media typically comprise documents, digital storage media, optical storage media, audio presentations, visual displays or the like, containing the information or instructions. For example, the communication medium may be a displayed web site, a kiosk, brochure, product label, package insert, advertisement, handout, public announcement audiotape, videotape, DVD, CD, computer-readable chip, computer-readable card, computer-readable disk, computer memory, or any combination thereof. Useful information includes one or more of (1) methods for promoting the health and wellness of animals and (2) contact information for the animal's caregivers to use if they have a question about the invention and its use. Useful instructions include techniques for using the probes, instructions for performing a gene expression assay, and administration amounts and frequency for the substances. The communication means is useful for instructing on the benefits of using the invention.

### EXAMPLES

Various aspects of the invention can be further illustrated by the following examples. It will be understood that these examples are provided merely for purposes of illustration and do not limit the scope of the invention disclosed herein unless otherwise specifically indicated.

### Example 1

This example briefly describes a study performed to test the ability of certain combinations of substances to mimic the life-extending effects of caloric restriction (CR) without reducing dietary intake. C57BL6 mice were fed a Control diet based on the AIN93M formula (American Institute of Nutrition (AIN) purified diet formula for maintenance of mature rodents) or a diet with similar nutrient composition but representing a 25% calorie restriction (CR)

Tissues were collected from mice on the Control diets at five and 25 months of age; tissues from nutrient-supplemented mice were collected at 25 months of age. RNA was isolated from tissues and gene expression changes were determined by qPCR using an Eppendorf “realplex2” instrument. Data for individual genes are set forth in certain of the subsequent examples.

### Example 2

This example describes the identification of biomarkers of aging in heart tissue.

The Affymetrix Mouse Genome 430 2.0 array was used to identify gene expression changes in heart of seven strains of mice (129, C57BL6, Balbc, C3H, CBA, DBA and B6C3HF1). A significant change in expression was determined using two-tailed t-tests for young vs. old mice (P<0.05, n=7 mice per strain per age group). Young mice were tested at 5 months of age, old mice were tested at 25 months of age. Table 1 shows the number of genes that were significantly changed in expression with age in each strain (P<0.05), and Table 2 lists the genes that were changed in expression in at least four of the seven strains.,

TABLE 1

| Strain | Number of Transcripts |
|---|---|
| 129 | 4,954 |
| B6 | 2,190 |
| Balbc | 2,137 |
| C3H | 3,236 |
| CBA | 1,118 |
| DBA | 1,521 |
| F1 | 1,287 |

TABLE 2

| Gene | | Entrez |
|---|---|---|
| Symbol | Gene Title | Gene |
| Adh1 | alcohol dehydrogenase 1 (class I) | 11522 |
| Agtrl1 | angiotensin receptor-like 1 | 23796 |
| Akr1b8 | aldo-keto reductase family 1, member B8 | 14187 |
| Aldh1a1 | aldehyde dehydrogenase family 1, subfamily A1 | 11668 |
| Alox5ap | arachidonate 5-lipoxygenase activating protein | 11690 |
| Amy1 | amylase 1, salivary | 11722 |
| Angptl2 | angiopoietin-like 2 | 26360 |
| Ankrd1 | ankyrin repeat domain 1 (cardiac muscle) | 107765 |
| Anxa1 | annexin A1 | 16952 |
| Apod | apolipoprotein D | 11815 |
| Apoe | apolipoprotein E | 11816 |
| Asah3l | N-acylsphingosine amidohydrolase 3-like | 230379 |
| Asph | aspartate-beta-hydroxylase | 65973 |
| Atp6v0e2 | ATPase, H+ transporting, lysosomal V0 subunit E2 | 76252 |
| Atp6v1c1 | ATPase, H+ transporting, lysosomal V1 subunit C1 | 66335 |
| Atp9a | ATPase, class II, type 9A | 11981 |
| Atxn10 | ataxin 10 | 54138 |
| BC023892 | cDNA sequence BC023892 | 212943 |
| Bckdhb | branched chain ketoacid dehydrogenase E1, beta polypeptide | 12040 |
| Bdh1 | 3-hydroxybutyrate dehydrogenase, type 1 | 71911 |
| Bre | brain and reproductive organ-expressed protein | 107976 |
| Bysl | bystin-like | 53414 |
| C1r | complement component 1, r subcomponent | 50909 |
| C3 | complement component 3 | 12266 |
| Cacna2d1 | calcium channel, voltage-dependent, alpha2/delta subunit 1 | 12293 |
| Camk2n1 | calcium/calmodulin-dependent protein kinase II inhibitor 1 | 66259 |
| Casq1 | calsequestrin 1 | 12372 |
| Ccdc72 | coiled-coil domain containing 72 | 66167 |
| Ccl6 | chemokine (C-C motif) ligand 6 | 20305 |
| Ccl8 | chemokine (C-C motif) ligand 8 | 20307 |
| Ccnd1 | cyclin D1 | 12443 |
| Cd163 | CD163 antigen | 93671 |
| Cdh22 | cadherin 22 | 104010 |
| Cebpd | CCAAT/enhancer binding protein (C/EBP), delta | 12609 |
| Cfb | complement factor B | 14962 |
| Chek2 | CHK2 checkpoint homolog (S. pombe) | 50883 |
| Churc1 | churchill domain containing 1 | 211151 |
| Cilp | cartilage intermediate layer protein, nucleotide pyrophosphohydrolase | 214425 |
| Ckb | creatine kinase, brain | 12709 |
| Clic5 | chloride intracellular channel 5 | 224796 |
| Col3a1 | procollagen, type III, alpha 1 | 12825 |
| Col8a1 | procollagen, type VIII, alpha 1 | 12837 |
| Cp | Ceruloplasmin | 12870 |
| Cpxm2 | carboxypeptidase X 2 (M14 family) | 55987 |
| Ctgf | connective tissue growth factor | 14219 |
| Ctss | cathepsin S | 13040 |
| Cxcl14 | chemokine (C—X—C motif) ligand 14 | 57266 |
| Cyb5r3 | cytochrome b5 reductase 3 | 109754 |
| Cyp27a1 | cytochrome P450, family 27, subfamily a, polypeptide 1 | 104086 |
| Dalrd3 | DALR anticodon binding domain containing 3 | 67789 |
| Dbnl | drebrin-like | 13169 |
| Dhrs1 | dehydrogenase/reductase (SDR family) member 1 | 52585 |
| Dhrs7c | dehydrogenase/reductase (SDR family) member 7C | 68460 |
| Dpep1 | dipeptidase 1 (renal) | 13479 |
| EG665317 | predicted gene, EG665317 | 665317 |
| Ehbp1l1 | EH domain binding protein 1-like 1 | 114601 |
| Ehmt2 | euchromatic histone lysine N-methyltransferase 2 | 110147 |
| Enpp2 | ectonucleotide pyrophosphatase/phosphodiesterase 2 | 18606 |
| Fads1 | fatty acid desaturase 1 | 76267 |
| Fbln2 | fibulin 2 | 14115 |
| Fcgr3 | Fc receptor, IgG, low affinity III | 14131 |
| Fez2 | fasciculation and elongation protein zeta 2 (zygin II) | 225020 |
| Fgfr1op2 | FGFR1 oncogene partner 2 | 67529 |
| Fkbp5 | FK506 binding protein 5 | 14229 |
| Fmo2 | flavin containing monooxygenase 2 | 55990 |
| Ftl2 | ferritin light chain 2 | 14337 |
| Fxyd6 | FXYD domain-containing ion transport regulator 6 | 59095 |
| Fzr1 | fizzy/cell division cycle 20 related 1 (Drosophila) | 56371 |
| Gcdh | glutaryl-Coenzyme A dehydrogenase | 270076 |
| Gda | guanine deaminase | 14544 |
| Gpm6b | glycoprotein m6b | 14758 |
| Hlx1 | H2.0-like homeo box 1 (Drosophila) | 15284 |
| Hod | homeobox only domain | 74318 |
| Hp1bp3 | heterochromatin protein 1, binding protein 3 | 15441 |
| Icam1 | intercellular adhesion molecule | 15894 |
| Ier3 | immediate early response 3 | 15937 |
| Ifit1 | interferon-induced protein with tetratricopeptide repeats 1 | 15957 |
| Il4ra | interleukin 4 receptor, alpha | 16190 |
| Isoc1 | Isochorismatase domain containing 1 | 66307 |
| Itm2a | integral membrane protein 2A | 16431 |
| Jph2 | junctophilin 2 | 59091 |
| Kbtbd2 | kelch repeat and BTB (POZ) domain containing 2 | 210973 |
| Kcnd2 | potassium voltage-gated channel, Shal-related family, member 2 | 16508 |
| Kcne1 | potassium voltage-gated channel, Isk-related subfamily, member 1 | 16509 |
| Klhdc1 | kelch domain containing 1 | 271005 |
| Lcn2 | lipocalin 2 | 16819 |
| Lect1 | leukocyte cell derived chemotaxin 1 | 16840 |
| Letm1 | leucine zipper-EF-hand containing transmembrane protein 1 | 56384 |
| Lgals3bp | lectin, galactoside-binding, soluble, 3 binding protein | 19039 |
| Lrp1 | low density lipoprotein receptor-related protein 1 | 16971 |
| Lrp11 | low density lipoprotein receptor-related protein 11 | 237253 |
| Ly6a | lymphocyte antigen 6 complex, locus A | 110454 |
| Man2a1 | mannosidase 2, alpha 1 | 17158 |
| Mef2a | myocyte enhancer factor 2A | 17258 |
| Mfge8 | milk fat globule-EGF factor 8 protein | 17304 |
| Mgp | matrix Gla protein | 17313 |
| Mier3 | mesoderm induction early response 1, family member 3 | 218613 |
| Mlf1 | myeloid leukemia factor 1 | 17349 |
| Mrc1 | mannose receptor, C type 1 | 17533 |
| Mt2 | metallothionein 2 | 17750 |
| Mybpc3 | myosin binding protein C, cardiac | 17868 |
| Myom2 | myomesin 2 | 17930 |
| Myot | Myotilin | 58916 |
| Ndrg4 | N-myc downstream regulated gene 4 | 234593 |
| Nfkbia | nuclear factor of kappa light chain gene enhancer in B-cells inhibitor, | 18035 |
| | alpha | |
| Npr3 | natriuretic peptide receptor 3 | 18162 |
| Nt5c2 | 5′-nucleotidase, cytosolic II | 76952 |
| Oas2 | 2′-5′ oligoadenylate synthetase 2 | 246728 |
| Osmr | oncostatin M receptor | 18414 |
| Pah | phenylalanine hydroxylase | 18478 |
| Pbxip1 | pre-B-cell leukemia transcription factor interacting protein 1 | 229534 |
| Pde1c | Phosphodiesterase 1C | 18575 |
| Pdlim4 | PDZ and LIM domain 4 | 30794 |
| Pgm5 | phosphoglucomutase 5 | 226041 |
| Phlda1 | pleckstrin homology-like domain, family A, member 1 | 21664 |
| Pkn2 | protein kinase N2 | 109333 |
| Pld3 | phospholipase D family, member 3 | 18807 |
| Plp2 | proteolipid protein 2 | 18824 |
| Postn | periostin, osteoblast specific factor | 50706 |
| Ppp1r3b | protein phosphatase 1, regulatory (inhibitor) subunit 3B | 244416 |
| Prg4 | proteoglycan 4 (megakaryocyte stimulating factor, articular | 96875 |
| | superficial zone protein) | |
| Prkar1a | protein kinase, cAMP dependent regulatory, type I, alpha | 19084 |
| Prkcq | protein kinase C, theta | 18761 |
| Ranbp5 | RAN binding protein 5 | 70572 |
| Rnf5 | ring finger protein 5 | 54197 |
| Rpl3l | ribosomal protein L3-like | 66211 |
| Rras | Harvey rat sarcoma oncogene, subgroup R | 20130 |
| Rtn2 | reticulon 2 (Z-band associated protein) | 20167 |
| Rtn4 | reticulon 4 | 68585 |
| Scn1b | sodium channel, voltage-gated, type I, beta | 20266 |
| Scn4b | sodium channel, type IV, beta | 399548 |
| Serpina3n | serine (or cysteine) peptidase inhibitor, clade A, member 3N | 20716 |
| Serpine2 | serine (or cysteine) peptidase inhibitor, clade E, member 2 | 20720 |
| Skap2 | src family associated phosphoprotein 2 | 54353 |
| Slc6a6 | solute carrier family 6 (neurotransmitter transporter, taurine), member 6 | 21366 |
| Snx10 | sorting nexin 10 | 71982 |
| Socs3 | suppressor of cytokine signaling 3 | 12702 |
| Srf | serum response factor | 20807 |
| Svep1 | sushi von Willebrand factor type A, EGF and pentraxin domain | 64817 |
| | containing 1 | |
| Tbc1d10c | TBC1 domain family, member 10c | 108995 |
| Tfpi | tissue factor pathway inhibitor | 21788 |
| Tgfb2 | transforming growth factor, beta 2 | 21808 |
| Tgm2 | transglutaminase 2, C polypeptide | 21817 |
| Thbs2 | thrombospondin 2 | 21826 |
| Thbs4 | thrombospondin 4 | 21828 |
| Timp2 | tissue inhibitor of metalloproteinase 2 | 21858 |
| Tln1 | talin 1 | 21894 |
| Tmem16k | transmembrane protein 16K | 102566 |
| Tmem176a | transmembrane protein 176A | 66058 |
| Tmem43 | transmembrane protein 43 | 74122 |
| Tnfaip8 | tumor necrosis factor, alpha-induced protein 8 | 106869 |
| Tomm40 | translocase of outer mitochondrial membrane 40 homolog (yeast) | 53333 |
| Tpte2 | transmembrane phosphoinositide 3-phosphatase and tensin homolog 2 | 57914 |
| Trim47 | tripartite motif protein 47 | 217333 |
| Tspan13 | tetraspanin 13 | 66109 |
| Tspan17 | tetraspanin 17 | 74257 |
| Uap1l1 | UDP-N-acteylglucosamine pyrophosphorylase 1-like 1 | 227620 |
| Ube2z | ubiquitin-conjugating enzyme E2Z (putative) | 268470 |
| Uchl1 | ubiquitin carboxy-terminal hydrolase L1 | 22223 |
| Vgll2 | vestigial like 2 homolog (Drosophila) | 215031 |
| Vwf | Von Willebrand factor homolog | 22371 |
| Wdr13 | WD repeat domain 13 | 73447 |
| Wisp2 | WNT1 inducible signaling pathway protein 2 | 22403 |
| Wtap | Wilms' tumour 1-associating protein | 60532 |
| Yipf7 | Yip1 domain family, member 7 | 75581 |
| Zadh2 | zinc binding alcohol dehydrogenase, domain containing 2 | 225791 |
| Zfp697 | zinc finger protein 697 | 242109 |

Sixteen potential markers of heart aging were selected for confirmation of array data by qPCR. Genes were selected based on multiple factors including (but not limited to): abundant expression in the microarray experiment, robust change in expression in the B6 strain, previous reports of gene associated with cardiac aging. Using the RNA samples from B6 used in the array study, qPCR analysis revealed that all 16 genes showed a change in expression with age. These genes are shown in Table 3. Of the 16 qPCR verified markers of heart aging, qPCR further revealed that the age-related expression pattern was reversed by CR in nine markers by at least about 32%. These nine markers are C3, Ccl8, Lcn2, Mt2, Pah, Prkcq, Serpina3n, Tmeml6k, and Vgll2.

TABLE 3

| Gene | | |
|---|---|---|
| Symbol | Gene Title | Entrez Gene |
| Amy1 | amylase 1, salivary | 11722 |
| Apod | apolipoprotein D | 11815 |
| Bdh1 | 3-hydroxybutyrate dehydrogenase, type 1 | 71911 |
| C3 | complement component 3 | 12266 |
| Casq1 | calsequestrin 1 | 12372 |
| Ccl8 | chemokine (C-C motif) ligand 8 | 20307 |
| Kcnd2 | potassium voltage-gated channel, | 16508 |
| | Shal-related family, member 2 | |
| Lcn2 | lipocalin 2 | 16819 |
| Mt2 | metallothionein 2 | 17750 |
| Myot | Myotilin | 58916 |
| Pah | phenylalanine hydroxylase | 18478 |
| Prkcq | protein kinase C, theta | 18761 |
| Serpina3n | serine (or cysteine) peptidase inhibitor, | 20716 |
| | clade A, member 3N | |
| Skap2 | src family associated phosphoprotein 2 | 54353 |
| Tmem 16k | transmembrane protein 16K | 102566 |
| Vgll2 | vestigial like 2 homolog (Drosophila) | 215031 |

The effects of the dietary interventions set forth in Example 1 on specific markers of heart aging are described below, along with reported function(s) of each marker.

### Stress Response

Metallothionein 2 (Mt2): Metallothionein genes are known to be induced in response to oxidative stress, and transgenic mice overexpressing human metallothionein 2A from a cardiac-specific promoter were protected from doxorubicin cardiotoxicity. Reports indicate that Mt2 can protect the heart from oxidative injury only if it is present before induction of oxidative stress. It should be noted that this gene was identified as a possible supermarker of skeletal muscle aging from the microarray data analysis, but a change in the expression of this gene was not confirmed by qPCR. In the heart, we observed an increase in the expression of this gene with age which was partially opposed by CR.

Apolipoprotein D (Apod): Apolipoprotein D is a member of the lipocalin family of genes and is involved in the immune and stress responses. Apod is induced in response to stress in the brain, and we previously identified this gene as a supermarker of mouse neocortex aging. In mouse heart, this gene was increased ˜2.5-fold with age, but the increase was not prevented by CR.

Lipocalin 2 (Lcn2). Numerous reports indicate that lipocalin 2 is induced by inflammatory and oxidative stress, and this increase is opposed in the presence of the reactive oxygen species scavengers cysteamine and DMSO. Therefore, Lcn2 could be a useful biomarker to identify oxidative stress both in vitro and in vivo. In mouse heart, this gene was increased nearly threefold with age, and the increase was entirely blunted by CR.

### Immune Response/Inflammation

Complement component 3 (C3): Complement component 3 plays a central role in the activation of complement system. Its activation is required for both classical and alternative complement activation pathways. We previously identified a closely related gene (C4) as a supermarker of aging in mouse skeletal muscle; these data are in agreement with many reports of increased immune activation with age. In mouse heart, C3 was nearly doubled in expression with age, and this increase was prevented by CR.

Chemokine (C-C motif) ligand 8 (Cc18): This cytokine displays chemotactic activity for monocytes, lymphocytes, basophils and eosinophils, and by recruiting leukocytes to sites of inflammation, this cytokine may contribute to tumor-associated leukocyte infiltration and to the antiviral state against HIV infection. In mouse heart, this gene was robustly increased (nearly sixfold) with age, and this increase was partially prevented by CR.

Protein kinase c theta (Prkcq): Protein kinase C (PKC) family members phosphorylate a wide variety of protein targets and are known to be involved in diverse cellular signaling pathways. Each member of the PKC family has a specific expression profile and is believed to play a distinct role. Prkcq is required for activation of T-lymphocytes; interestingly, a closely related gene (Prkcz) was increased in expression in skeletal muscle of multiple strains of mice, though this was not confirmed by qPCR. In heart, expression was increased nearly threefold with age and was slightly opposed by CR.

Serine (or cysteine) peptidase inhibitor, Glade A, member 3N (Serpina3n): This gene has been shown to suppress granzyme B-mediated apoptosis in cytotoxic T-lymphocytes, and the net effect of an increase in expression of this gene would be an inhibition of apoptosis of immune cells. In mouse heart, this gene was increased fourfold with age, and this increase was almost entirely prevented by CR. src family associated phosphoprotein 2 (Skap2): The protein encoded by this gene facilitates immune cell adhesion to sites of inflammation. In mouse heart, this gene was increased nearly twofold with age, and this increase was not affected by CR.

### Metabolism

3-hydroxybutyrate dehydrogenase, type 1 (Bdh1): This gene encodes a member of the short-chain dehydrogenase/reductase gene family and is involved in ketone body production by catalyzing the interconversion of acetoacetate and 3-hydroxybutyrate, the two major ketone bodies produced during fatty acid catabolism. In mouse heart, we observed a slight increase in the expression of this gene with age, which was further elevated by CR.

Phenylalanine hydroxylase (Pah): Pah encodes the enzyme phenylalanine hydroxylase that is the rate-limiting step in phenylalanine catabolism to tyrosine. Deficiency of this enzyme activity results in the autosomal recessive disorder phenylketonuria. In mouse heart, the expression of this gene was increased in expression nearly tenfold with age, and CR reduced the expression of this gene to about half of that seen in old controls.

### Heart Function

Amylase 1 (Amy1). Amylases are secreted proteins that hydrolyze 1,4-alpha-glucoside bonds in oligosaccharides and polysaccharides, and thus catalyze the first step in digestion of dietary starch and glycogen. The protein encoded by this gene may be related to heart function, as increased plasma levels of this protein have been reported in humans with chronic heart failure. We previously observed an increase in the expression of this gene in skeletal muscle of multiple strains of mice, though this gene was not identified as a supermarker in muscle. In mouse heart, the expression of this gene was increased ˜2.5-fold with age and was not markedly affected by CR.

Vestigial like 2 homolog (Drosophila) (Vgll2): This gene encodes a transcriptional cofactor that promotes skeletal muscle differentiation, so its expression in the heart is probably related to general cardiac muscle maintenance. In mouse heart, the expression of this gene was increased ˜7.5-fold with age, and CR prevented about half of the age-related increase in expression.

Myotilin (Myot): This gene encodes a protein found in the z-disc region of striated muscle and is involved in maintaining muscle structure and sarcomere organization. In humans, a mutation in this gene is associated with a form of muscular dystrophy. The expression of this gene was modestly increased with age in mouse heart, and the increase was not opposed by CR.

Calsequestrin (Casq1): Calsequestrin is the major calcium binding protein in the sarcoplasmic reticulum, and release of calcium ions bound to Casq1 results in muscle contraction. The expression of this gene was reduced in expression with age, possibly reflecting a general decline in muscle contraction with age. The expression of this gene was not changed by CR

Potassium voltage-gated channel, Sha1-related family, member 2 (Kcnd2): The protein encoded by this gene is responsible for outward potassium transport in the heart and expression of this gene is regulated by other genes sensitive to calcium status. The expression of this gene was decreased ˜25% with age, but was not changed by CR.

Transmembrane protein 16K (Tmem 16k): No functional data are available for this gene, however, the Gene Ontology consortium indicates that it is an integral membrane protein as inferred from electronic annotation. The expression of this gene was increased by ˜50% with age, and CR reduced the expression of this gene to below that seen in young control mice.

To gauge the overall effectiveness of an intervention designed to oppose age-related changes in gene expression, it was useful to generate an index which enables comparison of the effectiveness of an intervention in opposing the expression of the markers of heart aging. Below are describes two indices, though other analyses may also be devised. Each index is an average of the effect of a dietary intervention to oppose age-related changes in gene expression; the first index considers all 16 universal markers of cardiac aging described in this report; the second index considers only nine universal markers that were changed with both age and CR. For each gene, a “percent prevention” was calculated as the percent of the aging change that was opposed by an intervention. For example, a value of “100%” would indicate that the dietary intervention maintained the expression of a gene to the same level as seen in young controls. A prevention estimate greater than 100% would indicate the expression of a gene was shifted to a level that is “younger” than observed in young controls; conversely, a negative percent prevention would indicate that the expression of a gene was exacerbated beyond that seen in the old control group. The values for each gene are then averaged across a treatment, and the resulting index reveals the extent to which an intervention can oppose age-related changes in the expression of the markers of cardiac aging. For both indices, mild CR had the greatest ability to oppose age-related changes in the expression of the markers of heart aging.

### Example 3

This example describes the identification of transcriptional markers of aging in adipose tissue.

The Affymetrix Mouse Genome 430 2.0 array was used to identify gene expression changes in epididymal adipose tissue of seven strains of mice (129, C57BL6, Balbc, C3H, CBA, DBA and B6C3HF1).

A significant change in expression was determined using two-tailed t-tests for young vs. old mice (P<0.05, n=7 mice per strain per age group). Young mice were tested at five months of age, old mice were tested at 25 months of age. Table 4 shows the number of genes that were significantly changed with age in each strain, and Table 5 lists all genes that were changed in expression in at least five of the seven strains.

TABLE 4

| Strain | Number of Transcripts |
|---|---|
| 129 | 2,420 |
| B6 | 1,980 |
| Balbc | 1,646 |
| C3H | 5,391 |
| CBA | 5,993 |
| DBA | 3,788 |
| F1 | 4,187 |

TABLE 5

| Gene Symbol | Gene Title | Entrez Gene |
|---|---|---|
| Amotl1 | angiomotin-like 1 | 75723 |
| Cdc42ep1 | CDC42 effector protein (Rho GTPase binding) 1 | 104445 |
| Chkb | choline kinase beta | 12651 |
| AI597468 | expressed sequence AI597468 | 103266 |
| Flna | filamin, alpha | 192176 |
| Mst1r | macrophage stimulating 1 receptor (c-met-related tyr | 19882 |
| | kinase) | |
| Nmb | neuromedin B | 68039 |
| Nsd1 | nuclear receptor-binding SET-domain protein 1 | 18193 |
| Nap1l5 | nucleosome assembly protein 1-like 5 | 58243 |
| Otop1 | otopetrin 1 | 21906 |
| Osbpl8 | oxysterol binding protein-like 8 | 237542 |
| Pla2g2d | phospholipase A2, group IID | 18782 |
| Polr2d | polymerase (RNA) II (DNA directed) polypeptide D | 69241 |
| Pcdhb22 | protocadherin beta 22 | 93893 |
| 4921524J17Rik | RIKEN cDNA 4921524J17 gene | 66714 |
| Rc3h2 | ring finger and CCCH-type zinc finger domains 2 | 319817 |
| Rbms3 | RNA binding motif, single stranded interacting protein | 207181 |
| Akap2 | A kinase (PRKA) anchor protein 2 | 11641 |
| Aco2 | aconitase 2, mitochondrial | 11429 |
| Acsf3 | acyl-CoA synthetase family member 3 | 257633 |
| Acadl | acyl-Coenzyme A dehydrogenase, long-chain | 11363 |
| Acadvl | acyl-Coenzyme A dehydrogenase, very long chain | 11370 |
| Arf2 | ADP-ribosylation factor 2 | 11841 |
| Arl5b | ADP-ribosylation factor-like 5B | 75869 |
| Ankib1 | ankyrin repeat and IBR domain containing 1 | 70797 |
| Anxa2 | annexin A2 | 12306 |
| Aspn | Asporin | 66695 |
| Abcd4 | ATP-binding cassette, sub-family D (ALD), member 4 | 19300 |
| Bmp1 | bone morphogenetic protein 1 | 12153 |
| C1galt1c1 | C1GALT1-specific chaperone 1 | 59048 |
| Cidea | cell death-inducing DNA fragmentation factor, alpha | 12683 |
| | subunit-like effector A | |
| Coq9 | coenzyme Q9 homolog (yeast) | 67914 |
| Col5a2 | collagen, type V, alpha 2 | 12832 |
| Col6a1 | collagen, type VI, alpha 1 | 12833 |
| Col6a2 | collagen, type VI, alpha 2 | 12834 |
| Col18a1 | collagen, type XVIII, alpha 1 | 12822 |
| Clec4n | C-type lectin domain family 4, member n | 56620 |
| Crip2 | cysteine rich protein 2 | 68337 |
| Cyb5r1 | cytochrome b5 reductase 1 | 72017 |
| Cox4i1 | cytochrome c oxidase subunit IV isoform 1 | 12857 |
| Cox6c | cytochrome c oxidase, subunit VIc | 12864 |
| Dnmt3a | DNA methyltransferase 3A | 13435 |
| Dnaja3 | DnaJ (Hsp40) homolog, subfamily A, member 3 | 83945 |
| Emilin2 | elastin microfibril interfacer 2 | 246707 |
| Ear11 | eosinophil-associated, ribonuclease A family, member 11 | 93726 |
| Efemp1 | epidermal growth factor-containing fibulin-like | 216616 |
| | extracellular matrix protein 1 | |
| Fbxo6 | F-box protein 6 | 50762 |
| Fndc3b | fibronectin type III domain containing 3B | 72007 |
| Fyttd1 | forty-two-three domain containing 1 | 69823 |
| Gcnt2 | glucosaminyl (N-acetyl) transferase 2, I-branching | 14538 |
| | enzyme | |
| Gpd2 | glycerol phosphate dehydrogenase 2, mitochondrial | 14571 |
| Gnb4 | guanine nucleotide binding protein (G protein), beta 4 | 14696 |
| Hddc3 | HD domain containing 3 | 68695 |
| Hfe | Hemochromatosis | 15216 |
| Hadha | hydroxyacyl-Coenzyme A dehydrogenase/3-ketoacyl- | 97212 |
| | Coenzyme A thiolase/enoyl-Coenzyme A hydratase | |
| | (trifunctional protein), alpha subunit | |
| C130006E23 | Hypothetical protein C130006E23 | 331563 |
| Hif1a | hypoxia inducible factor 1, alpha subunit | 15251 |
| Itga6 | integrin alpha 6 | 16403 |
| Irf7 | interferon regulatory factor 7 | 54123 |
| Ldhb | lactate dehydrogenase B | 16832 |
| Lace1 | lactation elevated 1 | 215951 |
| Lxn | Latexin | 17035 |
| Lenep | lens epithelial protein | 57275 |
| Lims1 | LIM and senescent cell antigen-like domains 1 | 110829 |
| Macrod1 | MACRO domain containing 1 | 107227 |
| Msln | Mesothelin | 56047 |
| Myadm | myeloid-associated differentiation marker | 50918 |
| Gnptg | N-acetylglucosamine-1-phosphotransferase, gamma | 214505 |
| | subunit | |
| Ndufa10 | NADH dehydrogenase (ubiquinone) 1 alpha subcomplex | 67273 |
| | 10 | |
| Ndufa5 | NADH dehydrogenase (ubiquinone) 1 alpha subcomplex, 5 | 68202 |
| Ntn4 | netrin 4 | 57764 |
| Ntrk3 | neurotrophic tyrosine kinase, receptor, type 3 | 18213 |
| Nmnat3 | nicotinamide nucleotide adenylyltransferase 3 | 74080 |
| Nid1 | nidogen 1 | 18073 |
| Ogdh | oxoglutarate dehydrogenase (lipoamide) | 18293 |
| Ptcd3 | pentatricopeptide repeat domain 3 | 69956 |
| Ppic | peptidylprolyl isomerase C | 19038 |
| Pxdn | peroxidasin homolog (Drosophila) | 69675 |
| Kcnj15 | potassium inwardly-rectifying channel, subfamily J, | 16516 |
| | member 15 | |
| EG665378 | predicted gene, EG665378 | 665378 |
| Prss23 | protease, serine, 23 | 76453 |
| Pim1 | proviral integration site 1 | 18712 |
| Rmnd1 | required for meiotic nuclear division 1 homolog (S. cerevisiae) | 66084 |
| Rp2h | retinitis pigmentosa 2 homolog (human) | 19889 |
| Arhgef10 | Rho guanine nucleotide exchange factor (GEF) 10 | 234094 |
| Rhbdl3 | rhomboid, veinlet-like 3 (Drosophila) | 246104 |
| 1810013D10Rik | RIKEN cDNA 1810013D10 gene | 66278 |
| 2010005J08Rik | RIKEN cDNA 2010005J08 gene | 72046 |
| 2810482I07Rik | RIKEN cDNA 2810482I07 gene | 67243 |
| 4933439C20Rik | RIKEN cDNA 4933439C20 gene | 66776 |
| 9530058B02Rik | RIKEN cDNA 9530058B02 gene | 68241 |
| D830012I24Rik | RIKEN cDNA D830012I24 gene | 320070 |
| Sec23a | SEC23A (S. cerevisiae) | 20334 |
| Sema4a | sema domain, immunoglobulin domain (Ig), | 20351 |
| | transmembrane domain (TM) and short cytoplasmic | |
| | domain, (semaphorin) 4A | |
| Serpina3n | serine (or cysteine) peptidase inhibitor, clade A, member | 20716 |
| | 3N | |
| Serpinf1 | serine (or cysteine) peptidase inhibitor, clade F, member 1 | 20317 |
| Serhl | serine hydrolase-like | 68607 |
| LOC100047339 | similar to lysyl oxidase-like 2 | 100047339 |
| LOC100046998 | similar to optic atrophy 1 (autosomal dominant) | 100046998 |
| LOC100046740 | similar to Secreted acidic cysteine rich glycoprotein | 100046740 |
| Smcr7 | Smith-Magenis syndrome chromosome region, candidate | 237781 |
| | 7 homolog (human) | |
| Slc46a3 | solute carrier family 46, member 3 | 71706 |
| Slc6a13 | solute carrier family 6 (neurotransmitter transporter, | 14412 |
| | GABA), member 13 | |
| Stard3nl | STARD3 N-terminal like | 76205 |
| Sdhb | succinate dehydrogenase complex, subunit B, iron sulfur | 67680 |
| | (Ip) | |
| Suclg1 | succinate-CoA ligase, GDP-forming, alpha subunit | 56451 |
| Sucla2 | succinate-Coenzyme A ligase, ADP-forming, beta subunit | 20916 |
| Supv3l1 | suppressor of var1, 3-like 1 (S. cerevisiae) | 338359 |
| Syngr1 | synaptogyrin 1 | 20972 |
| Sycp3 | synaptonemal complex protein 3 | 20962 |
| Tspan2 | tetraspanin 2 | 70747 |
| Tanc1 | tetratricopeptide repeat, ankyrin repeat and coiled-coil | 66860 |
| | containing 1 | |
| Timm44 | translocase of inner mitochondrial membrane 44 | 21856 |
| Usp47 | ubiquitin specific peptidase 47 | 74996 |
| Uckl1 | uridine-cytidine kinase 1-like 1 | 68556 |
| Vps13a | vacuolar protein sorting 13A (yeast) | 271564 |
| Vash1 | vasohibin 1 | 238328 |
| Vcan | Versican | 13003 |
| Wwtr1 | WW domain containing transcription regulator 1 | 97064 |
| Zkscan17 | zinc finger with KRAB and SCAN domains 17 | 268417 |

Thirty-one potential markers of adipose tissue aging were selected for confirmation of array data by qPCR. In selecting candidate genes for validation by RT PCR, genes changed in all strains were given highest priority for further testing. Other considerations included avoiding genes with low expression (as judged by the average signal intensity from the microarray experiment) and avoiding genes that did not show change in expression by at least 50% (<1.5 or >1.5 fold change). Four genes that changed in all seven strains did not have a commercially available primer, and thus these genes could not be screened by qPCR. Beta actin (Actb) was not changed in any strain and served as the housekeeping gene for qPCR analyses.

For the remaining 27 genes identified as candidate markers of adipose tissue aging, qPCR analysis was used to test samples from the nutrient feeding study of Example 1 for validation of an aging change and opposition of the aging change by mild CR. A statistically significant change in expression with age was observed for 13 of the 27 genes when analyzed by qPCR. These are shown in Table 6. Eight of these 13 genes were further determined to be changed by age, and at least about 33% of the aging change was prevented by CR; these eight genes are: Aspn, Col6a2, Crip2, Emilin2, Otop1, Pla2g2d, Rhbd3and Slc6a13.

TABLE 6

| Gene Symbol | Gene Title | Entrez Gene |
|---|---|---|
| Aspn | Asporin | 66695 |
| Clec4n | C-type lectin domain family 4, member n | 56620 |
| Col6a2 | Collagen, type VI, alpha 2 | 12834 |
| Col18a1 | Collagen, type XVIII, alpha 1 | 12822 |
| Cox8b | Cytochrome c oxidase, subunit VIIIb | 12869 |
| Crip2 | Cysteine rich protein 2 | 68337 |
| Ear11 | Eosinophil-associated, ribonuclease A | 93726 |
| | family, member 11 | |
| Emilin2 | Elastin microfibril interfacer 2 | 246707 |
| Otop1 | Otopetrin 1 | 21906 |
| Pla2g2d | Phospholipase A2, group IID | 18782 |
| Rhbdl3 | Rhomboid, veinlet-like 3 (Drosophila) | 246104 |
| Slc6a13 | Solute carrier family 6 (neurotransmitter | 14412 |
| | transporter, GABA), member 13 | |
| Sycp3 | Synaptonemal complex protein 3 | 20962 |

The functional significance of the markers of adipose tissue aging is discussed below.

### Growth Factor-Responsive Genes

Asporin (Aspn): Transforming growth factor beta (TGF beta) is a secreted protein that plays a role in maintenance of the extracellularmatrix (ECM) by regulating the expression of genes involved in cytoskeletal maintenance (1). Asporin is a component of the ECM, and expression of asporin has been shown to be induced by TGF beta (2) in articular cartilage; we observed a decrease in asporin expression (2.0 fold) in adipose tissue with age. Taken together, these data suggest a decline in the maintenance of the ECM with age in adipose tissue. This decline may be prevented by CR, as the age related decline in Aspn expression was almost completely prevented by CR (1.8 fold increase in Old CR vs. Old Control mice).

Cysteine rich protein 2 (Crip2): Although little is known about the function of Crip2, it appears to belong to a family of proteins involved in cytoskeletal remodeling (3), and as with asporin (above), Crip2 expression has been shown to be induced by TGF beta (4). The pattern of Crip2 expression was very similar to that observed with asporin, including a significant change in expression with age (2.3 fold) that was prevented by CR (2.0 fold).

Rhomboid, veinlet-like 3 (Rhbdl3): The protein encoded by the Rhbdl3 gene has been characterized as the most evolutionarily conserved cDNA of the Drosophila gene rho which is modulate by epidermal growth factor signaling, and it plays a role in neural development in mice. This gene was robustly decreased in expression with age and CR completely prevented the age related change in the expression of this gene (6.2 fold change with age and 7.2 fold increase with CR).

In summary, the above three genes represent universal markers of adipose tissue aging that are regulated by growth factors, the expression of which can be modulated by diet.

### Decrease in ECM Microfiber Assembly with Age

Collagen, type VI, alpha 2 (Col6a2): Collagen VI is a component of the extracellular matrix and mutations in the Col6a2 gene in humans are associated with several congenital myopathies because of disrupted microfiber formation (6, 7). We have previously shown in our study of universal markers of mouse skeletal muscle aging that several collagen genes (Col1a1, Col1a2 and Col3a1) are decreased in expression in with age, and that this decline is opposed by CR. A significant reduction (2.1 fold decrease) in Col6a2 in adipose tissue suggesting a decreased maintenance of the ECM with age due to decreased microfiber assembly.

Elastin microfibril interfacer 2 (Emilin2). Little is known about the function of Emilin2, although it is reported to be synthesized and located in the ECM. Emilin2 may play a role in cell death via the extrinsic apoptotic pathway, as binding of apoptotic factors to Emilin2 results in caspase activation. Alternatively, others have suggested that elevated serum levels of the protein encoded by this gene may be a biomarker of ovarian cancer. In any case, a significant reduction in Emilin2 expression was observed with age in adipose tissue (-2.1 fold change with age), and CR opposed ˜35% of the aging change.

### Increased Inflammation with Age

Phospholipase A2, group IID (Pla2g2d): Phospholipases A2 (PLA2) are well-known for their ability to mobilize fatty acids from phospholipids which are subsequently converted to proinflammatory prostaglandins and leukotrienes (11). Interestingly, elevated levels of PLA2 may have consequences beyond lipid mobilization, as increased extracellular PLA2 is associated with increased levels of the proinflammatory cytokines TNFa and interleukin 1 (12). Expression of the Pla2g2d gene increased 3.1 fold with age and was partially (but not significantly) prevented by CR (1.4 fold change in expression).

### Less Characterized Genes

Otopetrin 1 (Otop1): The only report regarding the function of Otop1 shows that it is important for the formation of otoconia, structures of the inner ear that are responsible for perception of gravity and acceleration (13). Because these structures are formed by mineralization of calcium carbonate, it is likely that the function of this gene in adipose tissue is related to calcium homeostasis. Otop1 expression was increased in expression 3.0 fold with age and was partially (but not significantly) opposed by CR,(1.5 fold change in expression).

Solute carrier family 6, member 13 (Slc6a13): There are no reports regarding the function of this gene in mice or humans. However, it can be inferred from a single study in rats (14) that the protein encoded by the Slc6a13 gene is involved in transport of the neurotransmitter gamma aminobutyric acid (GABA). Slc6a13 was decreased in expression (1.8 fold change) with age, and this was partially (but not significantly) opposed by CR (1.3 fold change relative to Old Controls).

To gauge the overall effectiveness of an intervention designed to oppose age-related changes in the universal markers of aging, it was useful to generate an index which allows one to compare how an intervention opposes the expression of the universal markers of aging. Accordingly, an “aging prevention index” was calculated to describe the average effect of an intervention on age related changes in the expression of the eight universal markers of adipose tissue aging.

For each of the eight universal markers of adipose tissue aging, a “percent prevention” was calculated as the percent of the aging change that was opposed by an intervention. For example, a value of “100%” would indicate that the dietary intervention maintained the expression of a gene at the same level as seen in young controls. A prevention estimate greater than 100% would indicate the expression of a gene was shifted to a level that is “younger” than observed in young controls; conversely, a negative percent prevention would indicate that the expression of a gene was exacerbated beyond that seen in the Old Control group. The values for each gene are then averaged across a treatment, and the resulting index reveals the overall extent to which an intervention can oppose age-related changes at the transcriptional level.

### Example 4

This example describes the identification of transcriptional markers of aging in brain tissue.

The Affymetrix Mouse Genome 430 2.0 array was used to identify gene expression changes in neocortex of six strains of mice (C57BL6, Balbc, C3H, CBA, DBA and B6C3HF1). A significant change in expression was determined using two-tailed t-tests for young versus old mice (P<0.05, n=7 mice per strain per age group). Young mice were tested at five months of age, old mice were tested at 25 months of age.

Table 7 shows the number of genes that were significantly changed with age in each strain. Table 8 lists all genes that were changed in expression in at least three of the six strains.

TABLE 7

| Strain | Number of Transcripts |
|---|---|
| B6 | 1,029 |
| Balbc | 415 |
| C3H | 2,151 |
| CBA | 1,542 |
| DBA | 1,604 |
| F1 | 1,957 |

TABLE 8

| Gene Symbol | Gene Title | Entrez Gene |
|---|---|---|
| Abca8a | ATP-binding cassette, sub-family A (ABC1), member 8a | 217258 |
| Adamtsl4 | ADAMTS-like 4 | 229595 |
| Agxt2l1 | alanine-glyoxylate aminotransferase 2-like 1 | 71760 |
| AI465270 | expressed sequence AI465270 | 102097 |
| AI838057 | expressed sequence AI838057 | 101160 |
| Ak3l1 | adenylate kinase 3 alpha-like 1 | 11639 |
| Alad | aminolevulinate, delta-, dehydratase | 17025 |
| Alkbh3 | alkB, alkylation repair homolog 3 (E. coli) | 69113 |
| Ankrd17 | ankyrin repeat domain 17 | 81702 |
| Ankrd39 | ankyrin repeat domain 39 | 109346 |
| Anln | anillin, actin binding protein (scraps homolog, Drosophila) | 68743 |
| Anxa4 | annexin A4 | 11746 |
| Anxa5 | annexin A5 | 11747 |
| Apod | apolipoprotein D | 11815 |
| Arid4a | AT rich interactive domain 4A (Rbp1 like) | 238247 |
| Arl2 | ADP-ribosylation factor-like 2 | 56327 |
| Arpc1b | actin related protein 2/3 complex, subunit 1B | 11867 |
| Aspa | aspartoacylase (aminoacylase) 2 | 11484 |
| Atp2b1 | ATPase, Ca++ transporting, plasma membrane 1 | 67972 |
| B2m | beta-2 microglobulin | 12010 |
| BC035295 | cDNA sequence BC035295 | 207785 |
| BC061194 | cDNA sequence BC061194 | 381350 |
| Bcor | Bcl6 interacting corepressor | 71458 |
| Bid | BH3 interacting domain death agonist | 12122 |
| Bok | Bcl-2-related ovarian killer protein | 51800 |
| C030017B01Rik | RIKEN cDNA C030017B01 gene | 77524 |
| C130006E23 | Hypothetical protein C130006E23 | 331563 |
| C1qa | complement component 1, q subcomponent, alpha polypeptide | 12259 |
| C1qb | complement component 1, q subcomponent, beta polypeptide | 12260 |
| C1qc | complement component 1, q subcomponent, C chain | 12262 |
| C330006P03Rik | RIKEN cDNA C330006P03 gene | 320588 |
| Calb2 | calbindin 2 | 12308 |
| Camsap1l1 | calmodulin regulated spectrin-associated protein 1-like 1 | 67886 |
| Capns1 | calpain, small subunit 1 | 12336 |
| Cast | Calpastatin | 12380 |
| Ccnd2 | cyclin D2 | 12444 |
| Ccs | copper chaperone for superoxide dismutase | 12460 |
| Cd14 | CD14 antigen | 12475 |
| Cd52 | CD52 antigen | 23833 |
| Cd59a | CD59a antigen | 12509 |
| Cd68 | CD68 antigen | 12514 |
| Cd74 | CD74 antigen (invariant polypeptide of major histocompatibility | 16149 |
| | complex, class II antigen-associated) | |
| Cd81 | CD 81 antigen | 12520 |
| Cd9 | CD9 antigen | 12527 |
| Cdh11 | cadherin 11 | 12552 |
| Cdh8 | cadherin 8 | 12564 |
| Cdkl2 | cyclin-dependent kinase-like 2 (CDC2-related kinase) | 53886 |
| Cebpd | CCAAT/enhancer binding protein (C/EBP), delta | 12609 |
| Cend1 | cell cycle exit and neuronal differentiation 1 | 57754 |
| Chkb | choline kinase beta | 12651 |
| Cited1 | Cbp/p300-interacting transactivator with Glu/Asp-rich carboxy- | 12705 |
| | terminal domain 1 | |
| Clec7a | C-type lectin domain family 7, member a | 56644 |
| Cmtm3 | CKLF-like MARVEL transmembrane domain containing 3 | 68119 |
| Cmtm5 | CKLF-like MARVEL transmembrane domain containing 5 | 67272 |
| Cnksr2 | connector enhancer of kinase suppressor of Ras 2 | 245684 |
| Cnn3 | calponin 3, acidic | 71994 |
| Cntn6 | contactin 6 | 53870 |
| Commd9 | COMM domain containing 9 | 76501 |
| Comtd1 | catechol-O-methyltransferase domain containing 1 | 69156 |
| Cope | coatomer protein complex, subunit epsilon | 59042 |
| Coro1b | coronin, actin binding protein 1B | 23789 |
| Crim1 | cysteine rich transmembrane BMP regulator 1 (chordin like) | 50766 |
| Cst7 | cystatin F (leukocystatin) | 13011 |
| Ctsd | cathepsin D | 13033 |
| Ctss | cathepsin S | 13040 |
| Ctsz | cathepsin Z | 64138 |
| Cyb5r1 | cytochrome b5 reductase 1 | 72017 |
| Cyba | cytochrome b-245, alpha polypeptide | 13057 |
| Cyp20a1 | cytochrome P450, family 20, subfamily A, polypeptide 1 | 77951 |
| Cyp27a1 | cytochrome P450, family 27, subfamily a, polypeptide 1 | 104086 |
| D12Ertd647e | DNA segment, Chr 12, ERATO Doi 647, expressed | 52668 |
| D19Wsu12e | DNA segment, Chr 19, Wayne State University 12, expressed | 226090 |
| D1Ertd471e | DNA segment, Chr 1, ERATO Doi 471, expressed | 27877 |
| Ddc | dopa decarboxylase | 13195 |
| Ddt | D-dopachrome tautomerase | 13202 |
| Dhrs1 | dehydrogenase/reductase (SDR family) member 1 | 52585 |
| Diap2 | diaphanous homolog 2 (Drosophila) | 54004 |
| Dnalc1 | dynein, axonemal, light chain 1 | 105000 |
| Dusp6 | dual specificity phosphatase 6 | 67603 |
| E330009J07Rik | RIKEN cDNA E330009J07 gene | 243780 |
| Efha1 | EF hand domain family A1 | 68514 |
| EG434128 | predicted gene, EG434128 | 434128 |
| Egr1 | early growth response 1 | 13653 |
| Egr3 | early growth response 3 | 13655 |
| Epha4 | Eph receptor A4 | 13838 |
| Ephx1 | epoxide hydrolase 1, microsomal | 13849 |
| Ero1lb | ERO1-like beta (S. cerevisiae) | 67475 |
| Eya4 | eyes absent 4 homolog (Drosophila) | 14051 |
| Fand2a | fumarylacetoacetate hydrolase domain containing 2A | 68126 |
| Fbxo39 | F-box protein 39 | 327959 |
| Fcer1g | Fc receptor, IgE, high affinity I, gamma polypeptide | 14127 |
| Fcgr3 | Fc receptor, IgG, low affinity III | 14131 |
| Fnbp1l | formin binding protein 1-like | 214459 |
| Fndc3b | fibronectin type III domain containing 3B | 72007 |
| Foxg1 | forkhead box G1 | 15228 |
| Fxyd1 | FXYD domain-containing ion transport regulator 1 | 56188 |
| Gabrb3 | gamma-aminobutyric acid (GABA-A) receptor, subunit beta 3 | 14402 |
| Gal | Galanin | 14419 |
| Gatm | glycine amidinotransferase (L-arginine: glycine | 67092 |
| | amidinotransferase) | |
| Gda | guanine deaminase | 14544 |
| Gfap | glial fibrillary acidic protein | 14580 |
| Golph2 | golgi phosphoprotein 2 | 105348 |
| Gprin3 | GPRIN family member 3 | 243385 |
| Gpx3 | glutathione peroxidase 3 | 14778 |
| Grhpr | glyoxylate reductase/hydroxypyruvate reductase | 76238 |
| Gria3 | glutamate receptor, ionotropic, AMPA3 (alpha 3) | 53623 |
| Gstm1 | glutathione S-transferase, mu 1 | 14862 |
| Gstm6 | glutathione S-transferase, mu 6 | 14867 |
| Gstm7 | glutathione S-transferase, mu 7 | 68312 |
| Gsto1 | glutathione S-transferase omega 1 | 14873 |
| Gstt3 | glutathione S-transferase, theta 3 | 103140 |
| Gtf2f1 | general transcription factor IIF, polypeptide 1 | 98053 |
| H2-D1 | histocompatibility 2, D region locus 1 | 14964 |
| H2-M3 | histocompatibility 2, M region locus 3 | 14991 |
| Hapln2 | hyaluronan and proteoglycan link protein 2 | 73940 |
| Hebp2 | heme binding protein 2 | 56016 |
| Hectd2 | HECT domain containing 2 | 226098 |
| Helz | helicase with zinc finger domain | 78455 |
| Homer1 | homer homolog 1 (Drosophila) | 26556 |
| Hsd17b11 | hydroxysteroid (17-beta) dehydrogenase 11 | 114664 |
| Hsd3b7 | hydroxy-delta-5-steroid dehydrogenase, 3 beta- and steroid delta- | 101502 |
| | isomerase 7 | |
| Idh2 | isocitrate dehydrogenase 2 (NADP+), mitochondrial | 269951 |
| Ier3 | immediate early response 3 | 15937 |
| Ifit3 | interferon-induced protein with tetratricopeptide repeats 3 | 15959 |
| Ifitm3 | interferon induced transmembrane protein 3 | 66141 |
| Ikbkg | inhibitor of kappaB kinase gamma | 16151 |
| Il33 | interleukin 33 | 77125 |
| Imp4 | IMP4, U3 small nucleolar ribonucleoprotein, homolog (yeast) | 27993 |
| Itih3 | inter-alpha trypsin inhibitor, heavy chain 3 | 16426 |
| Jmjd1c | jumonji domain containing 1C | 108829 |
| Kcnf1 | potassium voltage-gated channel, subfamily F, member 1 | 382571 |
| Kcnj16 | potassium inwardly-rectifying channel, subfamily J, member 16 | 16517 |
| Kcnv1 | potassium channel, subfamily V, member 1 | 67498 |
| Klf10 | Kruppel-like factor 10 | 21847 |
| Klf9 | Kruppel-like factor 9 | 16601 |
| Klk6 | kallikrein related-peptidase 6 | 19144 |
| Lcat | lecithin cholesterol acyltransferase | 16816 |
| Lcp1 | lymphocyte cytosolic protein 1 | 18826 |
| Lefty1 | left right determination factor 1 | 13590 |
| Lgals3 | lectin, galactose binding, soluble 3 | 16854 |
| Lgals8 | lectin, galactose binding, soluble 8 | 56048 |
| Lhfp | lipoma HMGIC fusion partner | 108927 |
| LOC629605 | hypothetical protein LOC629605 | 629605 |
| LOC672274 | similar to Transcription factor SOX-4 | 672274 |
| Loh11cr2a | loss of heterozygosity, 11, chromosomal region 2, gene A | 67776 |
| | homolog (human) | |
| Lyzs | Lysozyme | 17105 |
| M6prbp1 | mannose-6-phosphate receptor binding protein 1 | 66905 |
| Malat1 | Metastasis associated lung adenocarcinoma transcript 1 (non- | 72289 |
| | coding RNA) | |
| Marcks | myristoylated alanine rich protein kinase C substrate | 17118 |
| Mccc2 | methylcrotonoyl-Coenzyme A carboxylase 2 (beta) | 78038 |
| Mdk | Midkine | 17242 |
| Mfhas1 | malignant fibrous histiocytoma amplified sequence 1 | 52065 |
| Mrpl10 | mitochondrial ribosomal protein L10 | 107732 |
| Mysm1 | myb-like, SWIRM and MPN domains 1 | 320713 |
| Nfyb | nuclear transcription factor-Y beta | 18045 |
| Nol4 | nucleolar protein 4 | 319211 |
| Ntsr2 | neurotensin receptor 2 | 18217 |
| Optn | Optineurin | 71648 |
| Pank3 | pantothenate kinase 3 | 211347 |
| Pcdh17 | protocadherin 17 | 219228 |
| Pcdh8 | protocadherin 8 | 18530 |
| Pcdhb2 | protocadherin beta 2 | 93873 |
| Pcdhb21 | protocadherin beta 21 | 93892 |
| Pcdhb9 | protocadherin beta 9 | 93880 |
| Pcolce2 | procollagen C-endopeptidase enhancer 2 | 76477 |
| Peci | peroxisomal delta3, delta2-enoyl-Coenzyme A isomerase | 23986 |
| Phlda1 | pleckstrin homology-like domain, family A, member 1 | 21664 |
| Plek | Pleckstrin | 56193 |
| Plekhf1 | pleckstrin homology domain containing, family F (with FYVE | 72287 |
| | domain) member 1 | |
| Plxna2 | plexin A2 | 18845 |
| Pmp22 | peripheral myelin protein | 18858 |
| Ppap2c | phosphatidic acid phosphatase type 2c | 50784 |
| Ppm1d | protein phosphatase 1D magnesium-dependent, delta isoform | 53892 |
| Ppp5c | protein phosphatase 5, catalytic subunit | 19060 |
| Prdm5 | PR domain containing 5 | 70779 |
| Psmb8 | proteosome (prosome, macropain) subunit, beta type 8 (large | 16913 |
| | multifunctional peptidase 7) | |
| Psmb9 | proteosome (prosome, macropain) subunit, beta type 9 (large | 16912 |
| | multifunctional peptidase 2) | |
| Ptpn12 | protein tyrosine phosphatase, non-receptor type 12 | 19248 |
| Pvrl3 | poliovirus receptor-related 3 | 58998 |
| Rab11fip2 | RAB11 family interacting protein 2 (class I) | 74998 |
| Rabl3 | RAB, member of RAS oncogene family-like 3 | 67657 |
| Rcbtb2 | regulator of chromosome condensation (RCC1) and BTB (POZ) | 105670 |
| | domain containing protein 2 | |
| Sbno1 | sno, strawberry notch homolog 1 (Drosophila) | 243272 |
| Shox2 | short stature homeobox 2 | 20429 |
| Slc14a1 | solute carrier family 14 (urea transporter), member 1 | 108052 |
| Slc15a4 | solute carrier family 15, member 4 | 100561 |
| Slit2 | slit homolog 2 (Drosophila) | 20563 |
| Slit3 | slit homolog 3 (Drosophila) | 20564 |
| Sord | sorbitol dehydrogenase | 20322 |
| Sos2 | Son of sevenless homolog 2 (Drosophila) | 20663 |
| Sparc | secreted acidic cysteine rich glycoprotein | 20692 |
| Spp1 | secreted phosphoprotein 1 | 20750 |
| Spred2 | sprouty-related, EVH1 domain containing 2 | 114716 |
| Ssbp3 | single-stranded DNA binding protein 3 | 72475 |
| Sstr4 | somatostatin receptor 4 | 20608 |
| St18 | suppression of tumorigenicity 18 | 240690 |
| Suhw4 | suppressor of hairy wing homolog 4 (Drosophila) | 235469 |
| Tcea2 | transcription elongation factor A (SII), 2 | 21400 |
| Th | tyrosine hydroxylase | 21823 |
| Tmbim1 | transmembrane BAX inhibitor motif containing 1 | 69660 |
| Tmem144 | transmembrane protein 144 | 70652 |
| Tmem176a | transmembrane protein 176A | 66058 |
| Tmem176b | transmembrane protein 176B | 65963 |
| Tnnt1 | troponin T1, skeletal, slow | 21955 |
| Tnnt2 | troponin T2, cardiac | 21956 |
| Trf | Transferring | 22041 |
| Tspan4 | tetraspanin 4 | 64540 |
| Tspo | translocator protein | 12257 |
| Ube2j1 | ubiquitin-conjugating enzyme E2, J1 | 56228 |
| Ubtd2 | ubiquitin domain containing 2 | 327900 |
| Vim | Vimentin | 22352 |
| Wwox | WW domain-containing oxidoreductase | 80707 |
| Zfp292 | zinc finger protein 292 | 30046 |
| Zmat5 | zinc finger, matrin type 5 | 67178 |
| Zswim6 | zinc finger, SWIM domain containing 6 | 67263 |
| Zwilch | Zwilch, kinetochore associated, homolog (Drosophila) | 68014 |

Eighteen potential markers of brain aging were selected for confirmation of array data by qPCR. Genes were selected based on multiple factors including but not limited to: abundant expression in the microarray experiment, robust change in expression in the B6 strain, previous reports of genes associated with brain aging. Using the RNA samples from B6 mice used in the array study, qPCR analysis revealed that 13/18 genes showed a change in expression with age. These 13 genes are shown in Table 9. Eight of the 13 genes were further determined to be changed by age and at least about 33% of the aging change was prevented by CR; these eight genes are: Apod, B2m, Clqa, Clqb, Ctsd, Gfap, Il33, Lyzs, and Spp1.

TABLE 9

| Gene | | |
|---|---|---|
| Symbol | Gene Title | Entrez Gene |
| Apod | apolipoprotein D | 11815 |
| B2m | beta-2 microglobulin | 12010 |
| C1qa | complement component 1, q subcomponent, alpha | 12259 |
| | polypeptide | |
| C1qb | complement component 1, q subcomponent, beta | 12260 |
| | polypeptide | |
| Cd68 | CD68 antigen | 12514 |
| Clec7a | C-type lectin domain family 7, member a | 56644 |
| Cst7 | cystatin F (leukocystatin) | 13011 |
| Ctsd | cathepsin D | 13033 |
| Gfap | glial fibrillary acidic protein | 14580 |
| Il33 | interleukin 33 | 77125 |
| Lgals3 | lectin, galactose binding, soluble 3 | 16854 |
| Lyzs | lysozyme | 17105 |
| Spp1 | secreted phosphoprotein 1 | 20750 |

The markers of brain aging identified above were used to asses the efficacy of CR to oppose age-related changes in brain aging. The extent to which the change in expression with age was opposed by CR (“percent prevention”) was determined using the indices described in the previous examples.

Many genes previously reported to be biomarkers of aging were identified in the array study and confirmed by qPCR, including Cd68, Ctsd and Gfap. CR opposed the change in expression for Ctsd, but was only moderately effective for Cd68 and Gfap.

Several genes were identified that have a neuroprotective action when expressed acutely, but have a detrimental effect when overexpressed on a chronic timescale (e.g., aging). Some of those genes include Apod, Cblqa and Clqb. A mild CR diet opposed the changes in Apod. The increase in the expression of Clqb seen with age was opposed by CR and in all supplemented mice.

There was a marked increase in the expression of genes involved in the immune and inflammatory responses (e.g., B2m, Clec71, Cst7, 1133, Lgals3). This is in strong agreement with previous reports of increased neuroinflammation with age. CR has been reported to oppose age-related changes in the expression of neuroinflammatory genes, though CR appeared to only oppose the age-related increase in B2m expression. Overall, the changes in the expression of inflammatory supermarkers was large (especially Clec7a and Cst7) and resistant to dietary intervention in this study. However, a larger restriction of caloric intake (˜40%) has been show to oppose the age-related changes in neuroinflammatory genes, thus alternative interventions have the potential to oppose age-related changes in these supermarkers.

Finally, many of the markers have been associated with neurodegenerative disorders such as Alzheimer's diseases (e.g., Apod, Clqa, Clqb, Ctsd, Lyzs, Spp1). Calorie restriction has been proposed to oppose the progression of Alzheimer's disease in humans and in mouse disease models. Accordingly, the mild CR used in this study opposed age-related changes in many of these genes.

Background information for reported or proposed functions of genes represented by the markers is set forth below.

Apod: Increased Apolipoprotein D expression has been reported in various neurological disorders, including Alzheimer's disease, schizophrenia, and stroke, and in the aging brain. Apod may be a stress response protein, since overexpression of this gene in Drosophila has been reported to lead to neuroprotection and lifespan extension. Thus, the level of Apod correlates with the level of endogenous stress.

B2M: beta 2-microglobulin (part of the class I major histocompatibility complex molecules) is also a reported marker of inflammation, and has previously been shown to increase with age in the cerebro-spinal fluid of aged humans and in the Parkinsonian brain.

Clqa and Clqb: These genes are reportedly involved in innate immunity and are markers of inflammation that we have previously shown to be activated with age in the mouse brain, and have also been shown to be activated in human neurodegenerative diseases such as Alzheimer's disease.

Cd68: Also known as macrosialin, Cd68 is a macrophage-specific protein, is reportedly increased by aging in selected brain regions of male C57BL/6NNia mice, and is thought to be expressed in microglia.

Clec7a: Also know as Dectin-1, this receptor reportedly can induce a variety of cellular responses in macrophages, including phagocytosis, the respiratory burst and cytokine production. This gene encodes a member of the C-type lectin/C-type lectin-like domain (CTL/CTLD) superfamily. The encoded glycoprotein is a small type II membrane receptor with an extracellular C-type lectin-like domain fold and a cytoplasmic domain with an immunoreceptor tyrosine-based activation motif. It reportedly functions as a pattern-recognition receptor that recognizes a variety of beta-1,3-linked and beta-1,6-linked glucans from fungi and plants, and in this way plays a role in innate immune response. Alternate transcriptional splice variants, encoding different isoforms, have been characterized. This gene is closely linked to other CTL/CTLD superfamily members on chromosome 12p13 in the natural killer gene complex region.

Cst7: Cystatin F is a glycosylated human low molecular weight cysteine proteinase inhibitor. Cystatins are important natural cysteine protease inhibitors targeting primarily papain-like cysteine proteases, including cathepsins and parasitic proteases like cruzipain, but also mammalian asparaginyl endopeptidase. Mammalian cystatin F, which is expressed almost exclusively in hematopoietic cells and accumulates in lysosome-like organelles, has been implicated in the regulation of antigen presentation and other immune processes. It is an unusual cystatin superfamily member with a reported redox-regulated activation mechanism and a restricted specificity profile.

Ctsd: Cathepsin D is a major lysosomal protease, and mutations in Ctsd that render it enzymatically defective have been reported recently in subsets of neuronal ceroid lipofuscinosces/Batten disease. The disease phenotype does not require Bax-mediated apoptosis, and instead appears to be mediated through autophagy. Cathepsin D-mediated proteolysis of apolipoprotein E may have a role in Alzheimer's disease. Up-regulation of the lysosomal system in experimental models of neuronal injury has also been reported.

Gfap: Glial fibrillary acidic protein is a classical marker of astrocyte activation, and probably the most well established brain aging marker. This gene encodes one of the major intermediate filament proteins of mature astrocytes. It is used as a marker to distinguish astrocytes from other glial cells during development. Mutations in this gene cause Alexander disease, a rare disorder of astrocytes in the central nervous system.

Il33: A poorly characterized cytokine, IL-33 is reported to be a dual function protein that may function as both a proinflammatory cytokine and an intracellular nuclear factor with transcriptional regulatory properties.

Lgals3: Galectin-3 is a multi-functional protein and reportedly participates in mediating inflammatory reactions. Galectin-3 is reportedly upregulated in microglial cells. Interestingly, Galectin-3 also reportedly promotes neural cell adhesion and neurite growth.

Lyzs: Also known as lysozyme, it is a poorly characterized and presumably lysosomal protein, which is abundant in leukocytes and is involved in inflammatory reactions.

Spp1: Secreted phosphoprotein-1, also known as osteopontin, is a secreted arginine-glycine-aspartate (RGD)-containing phosphoprotein. Spp1 appears to be overexpressed in Parkinson's disease. Osteopontin (OPN) has been implicated in inflammatory and wound-healing processes, including autoimmune uveitis.

### Example 5

This example describes the identification of transcriptional markers of aging in muscle tissue. To generate a panel of genes likely to be robust markers of age in mouse skeletal muscle, transcriptional profiling was performed using the Affymetrix Mouse Genome 430 2.0 Array on gastrocnemius muscle from seven mouse strains: 129/J, C57BL/6, CBA/J, DBA2J, C3H/HeJ, Balb/c, and B6C3HF1. Profiling was performed in seven young (five months of age) and seven old (28-30 months) mice from each strain. Two-tailed t-tests were performed to test for statistical significance of the change in gene expression with age. Of the ˜21,000 transcripts that were represented on the array, 172 transcripts were significantly (P<0.05) changed with age in at least six of the seven strains (Table 10).

TABLE 10

| | | Entrez |
|---|---|---|
| Gene Symbol | Gene Title | Gene |
| Acsl6 | acyl-CoA synthetase long-chain family member 6 | 216739 |
| Actr1a | ARP1 actin-related protein 1 homolog A (yeast) | 54130 |
| Adcy2 | adenylate cyclase 2 | 210044 |
| Agtrl1 | angiotensin receptor-like 1 | 23796 |
| Akr1b8 | aldo-keto reductase family 1, member B8 | 14187 |
| Aldh1a1 | aldehyde dehydrogenase family 1, subfamily A1 | 11668 |
| Amy1 | amylase 1, salivary | 11722 |
| Ankrd32 | ankyrin repeat domain 32 | 105377 |
| Antxr2 | anthrax toxin receptor 2 | 71914 |
| Apod | apolipoprotein D | 11815 |
| Arrdc4 | arrestin domain containing 4 | 66412 |
| Atp13a5 | ATPase type 13A5 | 268878 |
| AU018740 | expressed sequence AU018740 | 98528 |
| B3gnt1 | UDP-GlcNAc:betaGal beta-1,3-N-acetylglucosaminyltransferase 1 | 53625 |
| C4 | complement component 4 (within H-2S) | 12268 |
| Cd209b | CD209b antigen | 69165 |
| Cdkl2 | cyclin-dependent kinase-like 2 (CDC2-related kinase) | 53886 |
| Cdkn2c | cyclin-dependent kinase inhibitor 2C (p18, inhibits CDK4) | 12580 |
| Cds1 | CDP-diacylglycerol synthase 1 | 74596 |
| Ces2 /// | carboxylesterase 2 /// similar to carboxylesterase 2 | 234671 /// |
| LOC546098 | | 546098 |
| Chad | chondroadherin | 12643 |
| Chodl | chondrolectin | 246048 |
| Chrna1 | cholinergic receptor, nicotinic, alpha polypeptide 1 (muscle) | 11435 |
| Chrnb1 | cholinergic receptor, nicotinic, beta polypeptide 1 (muscle) | 11443 |
| Cilp2 | cartilage intermediate layer protein 2 | 68709 |
| Clybl | citrate lyase beta like | 69634 |
| Col11a1 | procollagen, type XI, alpha 1 | 12814 |
| Col1a1 | procollagen, type I, alpha 1 | 12842 |
| Col1a2 | procollagen, type I, alpha 2 | 12843 |
| Col3a1 | procollagen, type III, alpha 1 | 12825 |
| Col4a1 | procollagen, type IV, alpha 1 | 12826 |
| Col5a2 | procollagen, type V, alpha 2 | 12832 |
| Col6a1 | procollagen, type VI, alpha 1 | 12833 |
| Cpne2 | copine II | 234577 |
| Csprs | component of Sp100-rs | 114564 |
| Ctnnbl1 | catenin, beta like 1 | 66642 |
| Cuzd1 | CUB and zona pellucida-like domains 1 | 16433 |
| Cyt1 | cytokine like 1 | 231162 |
| Ddx6 | DEAD (Asp-Glu-Ala-Asp) box polypeptide 6 | 13209 |
| Dhx36 | DEAH (Asp-Glu-Ala-His) box polypeptide 36 | 72162 |
| Dmn | desmuslin | 233335 |
| Dmxl2 | Dmx-like 2 | 235380 |
| Dnajb14 | DnaJ (Hsp40) homolog, subfamily B, member 14 | 70604 |
| Dusp26 | dual specificity phosphatase 26 (putative) | 66959 |
| E030025D05Rik | RIKEN cDNA E030025D05 gene | 216613 |
| Edg2 | endothelial differentiation, lysophosphatidic acid G-protein- | 14745 |
| | coupled receptor, 2 | |
| Efha1 | EF hand domain family A1 | 68514 |
| Egfl6 | EGF-like-domain, multiple 6 | 54156 |
| Eif2c2 | eukaryotic translation initiation factor 2C, 2 | 239528 |
| Emb | embigin | 13723 |
| Fbxl4 | F-box and leucine-rich repeat protein 4 | 269514 |
| Fmo2 | flavin containing monooxygenase 2 | 55990 |
| Fmod | fibromodulin | 14264 |
| Fscn1 | fascin homolog 1, actin bundling protein (Strongylocentrotus) | 14086 |
| | purpuratus) | |
| Fut8 | fucosyltransferase 8 | 53618 |
| Gadd45a | growth arrest and DNA-damage-inducible 45 alpha | 13197 |
| Gca | grancalcin | 227960 |
| Gdap10 | ganglioside-induced differentiation-associated-protein 10 | 14546 |
| Gna14 | guanine nucleotide binding protein, alpha 14 | 14675 |
| Gtl2 /// Lphn1 | GTL2, imprinted maternally expressed untranslated mRNA /// | 17263///330814 |
| | latrophilin1 | |
| Hist1h3i | Histone 1, H3i (Hist1h3i), mRNA | 319153 |
| Hn1 | hematological and neurological expressed sequence 1 | 15374 |
| Homer2 | homer homolog 2 (Drosophila) | 26557 |
| Hook1 | hook homolog 1 (Drosophila) | 77963 |
| Hook3 | hook homolog 3 (Drosophila) | 320191 |
| Hsd17b7 | hydroxysteroid (17-beta) dehydrogenase 7 | 15490 |
| Icam1 | intercellular adhesion molecule | 15894 |
| Igh-6 | immunoglobulin heavy chain 6 (heavy chain of IgM) | 16019 |
| Itih5 | inter-alpha (globulin) inhibitor H5 | 209378 |
| Jarid1b | jumonji, AT rich interactive domain 1B (Rbp2 like) | 75605 |
| Jmjd3 | Jumonji domain containing 3, mRNA (cDNA clone | 216850 |
| | IMAGE: 4037702) | |
| Kcnab1 | potassium voltage-gated channel, shaker-related subfamily, beta | 16497 |
| | member 1 | |
| Kera | keratocan | 16545 |
| Krt1-18 | keratin complex 1, acidic, gene 18 | 16668 |
| Lgals3 | lectin, galactose binding, soluble 3 | 16854 |
| LOC241944 | similar to ZNF43 protein | 241944 |
| LOC545323 | similar to neurobeachin-like 1 | 545323 |
| Lpgat1 | lysophosphatidylglycerol acyltransferase 1 | 226856 |
| Lrp11 | low density lipoprotein receptor-related protein 11 | 237253 |
| Mfap4 | microfibrillar-associated protein 4 | 76293 |
| Mgst1 | microsomal glutathione S-transferase 1 | 56615 |
| Mia3 | melanoma inhibitory activity 3 | 338366 |
| Mll3 | myeloid/lymphoid or mixed-lineage leukemia 3 | 231051 |
| Mll5 | myeloid/lymphoid or mixed-lineage leukemia 5 | 69188 |
| Mt2 | metallothionein 2 | 17750 |
| Mtap7 | microtubule-associated protein 7 | 17761 |
| Myo5a | myosin Va | 17918 |
| Nipa1 | non imprinted in Prader-Willi/Angelman syndrome 1 homolog | 233280 |
| | (human) | |
| Nxn | nucleoredoxin | 18230 |
| Opcml | opioid binding protein/cell adhesion molecule-like | 330908 |
| Pb1 | PREDICTED: Mus musculus RIKEN cDNA 2310032M22 gene | 76748 |
| | (Pb1) | |
| Pcgf4 | polycomb group ring finger 4 | 12151 |
| Pdcd6ip | programmed cell death 6 interacting protein | 18571 |
| Pkp2 | plakophilin 2 | 67451 |
| Plekhb1 | pleckstrin homology domain containing, family B (evectins) | 27276 |
| | member 1 | |
| Plk2 | polo-like kinase 2 (Drosophila) | 20620 |
| Plp2 | proteolipid protein 2 | 18824 |
| Prkcz | protein kinase C, zeta | 18762 |
| Pura /// | purine rich element binding protein A /// RIKEN cDNA | 19290 /// |
| 6330411E07Rik | 6330411E07 gene | 70733 |
| Purb | Purine rich element binding protein B (Purb), mRNA | 19291 |
| Pvrl3 | poliovirus receptor-related 3 | 58998 |
| Rgs5 | regulator of G-protein signaling 5 | 19737 |
| Rhpn2 | rhophilin, Rho GTPase binding protein 2 | 52428 |
| Rnf125 | ring finger protein 125 | 67664 |
| Sbno1 | sno, strawberry notch homolog 1 (Drosophila) | 243272 |
| Serpina3n | serine (or cysteine) peptidase inhibitor, clade A, member 3N | 20716 |
| Serpinf1 | serine (or cysteine) peptidase inhibitor, clade F, member 1 | 20317 |
| Sh3bp5 | SH3-domain binding protein 5 (BTK-associated) | 24056 |
| Slc4a10 | solute carrier family 4, sodium bicarbonate cotransporter-like, | 94229 |
| | member10 | |
| Smc4l1 | SMC4 structural maintenance of chromosomes 4-like 1 (yeast) | 70099 |
| Spint2 | serine protease inhibitor, Kunitz type 2 | 20733 |
| Supt3h | suppressor of Ty 3 homolog (S. cerevisiae) | 109115 |
| Syt9 | synaptotagmin IX | 60510 |
| Taf9l | TAF9-like RNA polymerase II, TATA box binding protein (TBP)- | 407786 |
| | associated factor | |
| Tekt1 | tektin 1 | 21689 |
| Tnmd | tenomodulin | 64103 |
| Trim41 | tripartite motif-containing 41 | 211007 |
| Ttc14 | tetratricopeptide repeat domain 14 | 67120 |
| Ttc9c | tetratricopeptide repeat domain 9C | 70387 |
| Ubn1 | ubinuclein 1 | 170644 |
| Vsig4 | V-set and immunoglobulin domain containing 4 | 278180 |
| Vtn | vitronectin | 22370 |
| Wif1 | Wnt inhibitory factor 1 | 24117 |
| Zdhhc21 | zinc finger, DHHC domain containing 21 | 68268 |

Of the 172 transcripts that were identified in the microarray analysis, 21 genes were analyzed by qPCR to determine the change in expression with age and CR. Genes were chosen using several criteria—genes were selected based on having a known biological function, if they had a relatively abundant expression in the array study and/or if studies in peer-reviewed literature suggest a role in muscle aging. RT-PCR analysis was performed using an Applied Biosystems 7000 instrument using off-the-shelf PCR primers designed by Applied Biosystems. TATA box binding protein (Tbp) was used as an internal control for all RT-PCR analyses, as this gene was previously shown not to change with age or CR. Of the 21 genes that were tested, 13 genes showed a change in expression with age in at least six of the seven strains (Table 11). Of the 13 genes, eleven showed reversal by CR. These eleven genes are: C4, Cdkn2c, Cds1, Col1a1, Col1a2, Col3a1, Edg2, Igh-6, Mt2, Plk2, and Syt9.

TABLE 11

| Gene | | Entrez |
|---|---|---|
| Symbol | Gene Title | Gene |
| C4 | complement component 4 (within H-2S) | 12268 |
| Cdkn2c | cyclin-dependent kinase inhibitor 2C | 12580 |
| | (p18, inhibits CDK4) | |
| Cds1 | CDP-diacylglycerol synthase 1 | 74596 |
| Col1a1 | procollagen, type I, alpha 1 | 12842 |
| Col1a2 | procollagen, type I, alpha 2 | 12843 |
| Col3a1 | procollagen, type III, alpha 1 | 12825 |
| Dusp26 | dual specificity phosphatase 26 (putative) | 66959 |
| Edg2 | endothelial differentiation, lysophosphatidic acid | 14745 |
| | G-protein-coupled receptor, 2 | |
| Igh-6 | immunoglobulin heavy chain 6 (heavy chain of IgM) | 16019 |
| Mt2 | metallothionein 2 | 17750 |
| Plk2 | polo-like kinase 2 (Drosophila) | 20620 |
| Rhpn2 | rhophilin, Rho GTPase binding protein 2 | 52428 |
| Syt9 | synaptotagmin IX | 60510 |

Of the genes shown in Table 11 two can be broadly classified as being involved in inflammatory response (C4 and Igh6), two can be broadly classified in DNA damage/cell cycle checkpoint (Cdkn2c and Plk2), two can be classified in the stress response (Mt2 and Dusp26), one (Cds1) can be broadly classified as being involved in biosynthesis, one can be classified as involved in calcium metabolism (Syt9) and five genes (Colla1, Colla2, Col3a1, Edg2, Rhpn2) can be classified as being involved in cytoskeletal remodeling.

After an appropriate panel of biomarkers had been validated via qPCR in (changed with age and in most instances the change reversed by CR), the expression of those genes was analyzed in mRNA samples from the nutrient study of Example 1. Results pertaining to specific markers, along with a description of the reported functional significance of those markers, are discussed below.

Complement C4: The fourth component of the complement cascade is an essential factor in innate immunity. Its activation has been observed during normal brain aging in humans and also in Alzheimer's disease brain. Different alleles of C4 in humans have been linked in health and survival, suggesting that C4 status impacts health directly. Complement C4 has also been linked to autoimmune disease.

Igh-6: Igh6 is a B-cell antigen required for B-cell maturation. Igh6 deficient mice are commonly used as a model of B cell deficiency. Thus, increased Igh-6 in skeletal muscle with age may be secondary to an increase in B-cell infiltration in this tissue, which was completely prevented by CR in these studies.

Cdkn2C (p18): CdKn2C, also known as pl8INK4c, is a GI-phase cyclin kinase inhibitor (CKI). It is one of several CKIs involved in cell cycle arrest in response to DNA damage. CKI inhibitors are tumor suppressor genes, since mutations in either p18 or the related p16 lead to tumorigenesis. p16 in particular has been linked to cellular senescence of the adult stem cell population compartment. It is likely that the observed age-related p18 activation reflects accrued DNA damage.

Plk2: Polo-kinase 2 is a polo-like kinase expressed at G1 in cultured cells and specific animal tissues that functions in the DNA damage response. Polo, the founding member of this gene family, was identified in Drosophila and plays a role in the control of cell division.

Mt2: The metallothioneins (I, II and III) are low molecular weight, cysteine rich metal binding proteins found in a wide variety of organisms and known to be induced under a wide variety of stress conditions. Because these proteins control the traffic of intracellular zinc, it is thought that control of zinc levels through metallothioneins is an important aspect of the cellular defense against stress.

Dusp26: DUSPs (Dual-specificity tyrosine phosphtases) are similar to tyrosine phosphatases by possessing the tyrosine phosphatase signature domain (I/V)HCXAGXGR(S/T) involved in their catalytic activity. Many members of this class have been identified, including DUSP1-9, DUSP16 and DUSP-26, also known as MKP 8. All of these proteins dephosphorylate serine/threonine and tyrosine residues on different MAP kinase members leading to their inactivation. DUSPs are activated by stimuli that trigger MAP kinase pathways such as heat shock, mitogens and hypoxia. Dusp26 has recently been shown to associate with the heat shock transcriptional factor 4b, establishing a link between this DUSP and the heat shock response. Surprisingly, CR was unable to prevent the age-related induction of Dusp26

Cds1: CDP-diacylglycerol synthase is a rate limiting enzyme involved in glycerolipid biosynthesis, which serves as a precursor to both phosphoinositides and phosphatidylglycerol. It is thought that the CDP-diacylglycerol synthases regulate the activity of phospholipids biosynthetic pathways.

Edg2: The expression of Edg2 (endothelial differentiation, lysophosphatidic acid G-protein coupled receptor 2) decreased with age, and this decrease was almost entirely prevented by CR. Lysophosphatidic acid induces cytoskeletal rearrangement, and because Edg2 is a receptor for this molecule, these findings recapitulate the general pattern seen in this study that cytoskeletal remodeling is a common feature of aging in mouse skeletal muscle .

Colla1, Colla2, Col3a1: Colla and Colla2 encode the chains of type I procollagen. Mutations in these genes are associated with the human disease osteogenesis imperfecta. To our knowledge, this is the first discovery of a coordinated downregulation of collagen genes with aging. Three pro-collagen genes (Colla1, Colla2, Col3a1) showed changes with age and opposition by CR.

Rhpn2: Rhophilin 2 is a Rho GTPase binding protein. It has been postulated to play a role in endocytosis, and a two-hybrid yeast screen suggests that components of cytoskeleton are partners of rhophilin 2.

Syt9: Calcium influx into presynaptic nerve terminals and neuroendocrine cells triggers exocytosis of synaptic and secretory vesicles. Synaptotagmins, including Syt9 are calcium binding proteins that are thought to be calcium sensors for exocytosis.

The specification has disclosed typical preferred embodiments of the invention. Although specific terms are employed, they are used in a generic and descriptive sense only and not for purposes of limitation, the scope of the invention being set forth in the claims. Clearly, many modifications and variations of the invention are possible in light of the above teachings. It is therefore to be understood that within the scope of the appended claims the invention may be practiced otherwise than as specifically described.

1. A method of identifying gene expression markers of aging in a selected tissue comprising selecting one or more genes differentially expressed in the tissue in old subjects as compared with young subjects, using a criterion that the gene is differentially expressed in a multiplicity of strains, breeds or ethnic groups of a species at a pre-determined significance level.

2. The method of claim 1 wherein the selected gene is differentially expressed in three or more strains, breeds or ethnic groups.

3. The method of claim 1 wherein the selected gene is differentially expressed in five or more of the strains, breeds or ethnic groups.

4. The method of claim 1 wherein the selected gene is differentially expressed in at least 50% of the strains, breeds or ethnic groups tested.

5. The method of claim 1 wherein the selected gene is differentially expressed in at least 75% of the strains, breeds or ethnic groups tested.

6. The method of claim 1 wherein the tissue is heart, muscle, brain or adipose tissue.

7. (canceled)

8. The method of claim 1 further comprising a criterion that differential expression of the gene differentially expressed in old subjects as compared with young subjects is at least partially reversed by caloric restriction.

9. The method of claim 1 further comprising a criterion that the gene differentially expressed in old subjects as compared with young subjects is known or suspected to be associated with one or more aging-related physiological functions.

10. The method of claim 1 wherein the species is canine or feline.

11. A combination comprising a plurality of polynucleotides that are differentially expressed in a selected tissue in old subjects as compared with young subjects, wherein the selected tissue is heart, adipose, brain or muscle tissue and the polynucleotides are selected from genes encoding proteins listed in Table 2, Table 5, Table 8 or Table 10, or fragments thereof.

12. The combination of claim 11 wherein the selected tissue is heart and the polynucleotides are selected from genes encoding two or more of Amy1, Apod, Bdh1, C3, Casq1, Cc18, Kcnd2, Lcn2, Mt2, Myot, Pah, Prkcq, Serpina3n, Skap2, Tmeml6k, and Vgll2.

13. The combination of claim 12 wherein the differential expression is reversed by caloric restriction and the polynucleotides are selected from genes encoding two or more of C3, Cc18, Lcn2, Mt2, Pah, Prkcq, Serpina3n, Tmeml6k, and Vgll2.

14. The combination of claim 11 wherein the selected tissue is adipose and the polynucleotides are selected from genes encoding two or more of Aspn, Clec4n, Col6a2, Coll8a1, Cox8b, Crip2, Ear11, Emilin2, Otop1, Pla2g2d, Rhbd13, Slc6a13, and Sycp3.

15. The combination of claim 14 wherein the differential expression is reversed by caloric restriction and the polynucleotides are selected from genes encoding two or more of Aspn, Col6a2, Crip2, Emilin2, Otop1, Pla2g2d, Rhbd13, and Slc6a13.

16. The combination of claim 11 wherein the selected tissue is brain and the polynucleotides are selected from genes encoding two or more of Apod, B2m, Clqa, Clqb, Cd68, Clec7a, Cst7, Ctsd, Gfap, Il33, Lgals3, Lyzs, and Spp1.

17. The combination of claim 16 wherein the differential expression is reversed by caloric restriction and the polynucleotides are selected from genes encoding two or more of Apod, B2m, Clqa, Clqb, Ctsd, Gfap, Il33, Lyzs, and Spp1.

18. The combination of claim 11 wherein the selected tissue is muscle and the polynucleotides are selected from genes encoding two or more of C4, Cdkn2c, Cds1, Colla1, Colla2, Col3a1, Dusp26, Edg2, Igh-6, Mt2, Plk2, Rhpn2, and Syt9.

19. The combination of claim 18 wherein the differential expression is reversed by caloric restriction and the polynucleotides are selected from genes encoding two or more of C4, Cdkn2c, Cds1, Colla1, Colla2, Col3a1, Edg2, Igh-6, Mt2, Plk2, and Syt9.

20. The combination of claim 11 wherein the polynucleotides are canine or feline polynucleotides.

21. A composition comprising two or more probes for detecting differential gene expression in a selected tissue in old subjects as compared with young subjects, wherein the selected tissue is heart, adipose, brain or muscle tissue, and the probes comprise:
a) polynucleotides that specifically hybridize to two or more genes encoding proteins listed in Table 2, Table 5, Table 8 or Table 10, or fragments thereof; or
b) polypeptide binding agents that specifically bind to two or more polypeptides selected from proteins listed in Table 2, Table 5, Table 8 or Table 10, or fragments thereof.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)
